(19) United States
(12) Patent Application Publication
(10) Pub. No.: US 2024/0175062 A1
(43) Pub. Date: May 30, 2024

(54) LIPOXYGENASE-BASED RECOMBINANT MICROORGANISMS, AND METHOD FORPREPARING HYDROXY FATTY ACIDS AND SECONDARY FATTY ALCOHOLS USING SAME

(71) Applicants: EWHA UNIVERSITY - INDUSTRY COLLABORATION FOUNDATION, Seoul (KR); KONKUK UNIVERSITY INDUSTRIAL COOPERATION CORP, Seoul (KR)

(72) Inventors: Jin Byung Park, Seoul (KR); Jeong Hoo Lee, Seoul (KR); Hee Jeong Cha, Gyeongsangnam-do (KR); Jeong Hye Yoon, Seoul (KR); Deok Kun OH, Gyeonggi-do (KR)

(21) Appl. No.: 18/549,900

(22) PCT Filed: Mar. 10, 2022
(86) PCT No.: PCT/KR2022/003365

§ 371 Date: Sep. 9, 2023

(30) Foreign Application Priority Data
Mar. 10, 2021 (KR) 10-2021-0031571
Feb. 28, 2022 (KR) 10-2022-0026074

(51) Int. Cl. C12P 7/6409; C12N 9/02

(52) U.S. Cl. CPC C12P 7/6409; C12N 9/0069

(57) ABSTRACT

The present invention relates to: recombinant microorganisms expressing lipoxygenase; and recombinant microorganisms co-expressing lipoxygenase and Chlorella-derived decarboxylase. It was confirmed that when using lipoxygenase-based recombinant microorganisms according to the present invention as whole-cell biocatalysts, hydroxy fatty acids can be produced from unsaturated fatty acids. In addition, it was confirmed that when using the recombinant microorganisms co-expressing lipoxygenase and Chlorella-derived decarboxylase as whole-cell biocatalysts, secondary fatty alcohols can be produced from unsaturated fatty acids. Thus, the recombinant microorganisms constructed in the present invention can be used in various ways in the field of hydroxy fatty acid and secondary fatty alcohol production.

### REFERENCE TO AN ELECTRONIC SEQUENCE LISTING

The contents of the electronic sequence listing (OP-23087-PCT-US_SEQUENCE LISTING.pdf; Size: 45,232 bytes; and Date of Creation: Sep. 8, 2023) is herein incorporated by reference in its entirety.

### TECHNICAL FIELD

The present invention relates to: recombinant microorganisms expressing lipoxygenase; and recombinant microorganisms co-expressing lipoxygenase and Chlorella-derived decarboxylase.

### BACKGROUND ART

Renewable oil is one of the most important biological resources. The renewable oil includes vegetable oil, microalgal oil, oil produced by oleaginous yeasts, and the like. The renewable oil is also widely used in the production of oleochemicals for the chemical industry as well as biodiesels. The oleochemicals include fatty acids, fatty acid methyl esters, amines and alcohols, and are used in the manufacture of various chemicals such as surfactants, lubricants and coatings.

Hydroxy fatty acids have one or more hydroxyl groups in the central chain of general fatty acids, and secondary fatty alcohols are in the form with one or more hydroxyl groups in a general fatty chain, and are found in trace amounts mainly in plants in nature. The hydroxy fatty acids and the secondary fatty alcohols show specific properties such as high viscosity and reactivity by the hydroxyl groups attached to the fatty chain. The hydroxy fatty acids and the secondary fatty alcohols have specific properties by hydroxyl groups, and thus may have various physiologically active functions and functionalities, and as a result, may be widely applied to whole industry, such as new pesticides, new medicines, high-functional resins and textile substances, biodegradable plastic substances, lubricants, cosmetics, and paints.

Although various functions of the hydroxy fatty acids and the secondary fatty alcohols are known, the hydroxy fatty acids and the secondary fatty alcohols that exist in nature are known to be present only in trace amounts in plants. Accordingly, studies to produce hydroxy fatty acids and secondary fatty alcohols using microorganisms have been attempted.

### DISCLOSURE

### Technical Problem

Therefore, the present inventors have completed the present invention by constructing a lipoxygenase-based whole-cell catalyst system for preparing hydroxy fatty acids and secondary fatty alcohols using microorganisms.

An object of the present invention is to provide recombinant microorganisms including a lipoxygenase gene represented by a nucleotide sequence set forth in SEQ ID NO: 1 or 2.

Another object of the present invention is to provide a method for producing hydroxy fatty acids, including preparing hydroperoxy fatty acids by reacting the recombinant microorganisms with unsaturated fatty acids.

Yet another object of the present invention is to provide a composition for producing hydroxy fatty acids, including the recombinant microorganisms.

Yet another object of the present invention is to provide a method for preparing recombinant microorganisms for producing hydroxy fatty acids, including introducing a lipoxygenase gene represented by a nucleotide sequence set forth in SEQ ID NO: 1 or 2 into microorganisms.

Yet another object of the present invention is to provide recombinant microorganisms, including a lipoxygenase gene represented by a nucleotide sequence set forth in SEQ ID NO: 1 or 2; and a decarboxylase gene represented by a gene set forth in SEQ ID NO: 3.

Yet another object of the present invention is to provide a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

Yet another object of the present invention is to provide a composition for producing hydroxy fatty acids, including the recombinant microorganisms including the lipoxygenase gene and the decarboxylase gene.

Yet another object of the present invention is to provide a method for preparing recombinant microorganisms for producing hydroxy fatty acids or secondary fatty alcohols, including introducing a lipoxygenase gene represented by a nucleotide sequence set forth in SEQ ID NO: 1 or 2 and a decarboxylase gene represented by a nucleotide sequence set forth in SEQ ID NO: 3 into microorganisms.

Yet another object of the present invention is to provide recombinant microorganisms including a lipoxygenase gene represented by at least one nucleotide sequence selected from SEQ ID NOs: 8 to 10.

Yet another object of the present invention is to provide a method for producing hydroxy fatty acids, including preparing hydroperoxy fatty acids by reacting the recombinant microorganisms with unsaturated fatty acids.

Yet another object of the present invention is to provide a composition for producing hydroxy fatty acids, including the recombinant microorganisms.

Yet another object of the present invention is to provide a method for preparing recombinant microorganisms for producing hydroxy fatty acids, including introducing a lipoxygenase gene represented by at least one nucleotide sequence set forth in SEQ ID NOs: 8 to 10 into microorganisms.

Yet another object of the present invention is to provide recombinant microorganisms, including a lipoxygenase gene represented by a nucleotide sequence set forth in SEQ ID NOs: 8 to 10; and a decarboxylase gene represented by a gene set forth in SEQ ID NO: 11.

Yet another object of the present invention is to provide a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

Yet another object of the present invention is to provide a composition for producing hydroxy fatty acids, including the recombinant microorganisms including the lipoxygenase gene and the decarboxylase gene.

Yet another object of the present invention is to provide a method for preparing recombinant microorganisms for producing hydroxy fatty acids or secondary fatty alcohols, including introducing a lipoxygenase gene represented by a nucleotide sequence set forth in SEQ ID NOs: 8 to 10 and a decarboxylase gene represented by a nucleotide sequence set forth in SEQ ID NO: 11 into microorganisms.

### Technical Solution

In order to achieve the object, an aspect of the present invention provides recombinant microorganisms including a lipoxygenase gene represented by a nucleotide sequence set forth in SEQ ID NO: 1 or 2.

Another aspect of the present invention provides a method for producing hydroxy fatty acids, including preparing hydroperoxy fatty acids by reacting the recombinant microorganisms with unsaturated fatty acids.

Yet another aspect of the present invention provides a composition for producing hydroxy fatty acids, including the recombinant microorganisms.

Yet another aspect of the present invention provides a method for preparing recombinant microorganisms for producing hydroxy fatty acids, including introducing a lipoxygenase gene represented by a nucleotide sequence set forth in SEQ ID NO: 1 or 2 into microorganisms.

Yet another aspect of the present invention provides recombinant microorganisms, including a lipoxygenase gene represented by a nucleotide sequence set forth in SEQ ID NO: 1 or 2; and a decarboxylase gene represented by a gene set forth in SEQ ID NO: 3.

Yet another aspect of the present invention provides a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

Yet another aspect of the present invention provides a composition for producing hydroxy fatty acids, including the recombinant microorganisms including the lipoxygenase gene and the decarboxylase gene.

Yet another aspect of the present invention provides a method for preparing recombinant microorganisms for producing hydroxy fatty acids or secondary fatty alcohols, including introducing a lipoxygenase gene represented by a nucleotide sequence set forth in SEQ ID NO: 1 or 2 and a decarboxylase gene represented by a nucleotide sequence set forth in SEQ ID NO: 3 into microorganisms.

Yet another aspect of the present invention provides recombinant microorganisms including a lipoxygenase gene represented by at least one nucleotide sequence selected from SEQ ID NOs: 8 to 10.

Yet another aspect of the present invention provides a method for producing hydroxy fatty acids, including preparing hydroperoxy fatty acids by reacting the recombinant microorganisms with unsaturated fatty acids.

Yet another aspect of the present invention provides a composition for producing hydroxy fatty acids, including the recombinant microorganisms.

Yet another aspect of the present invention provides a method for preparing recombinant microorganisms for producing hydroxy fatty acids, including introducing a lipoxygenase gene represented by at least one nucleotide sequence selected from SEQ ID NOs: 8 to 10 into microorganisms.

Yet another aspect of the present invention provides recombinant microorganisms, including a lipoxygenase gene represented by a nucleotide sequence set forth in SEQ ID NOs: 8 to 10; and a decarboxylase gene represented by a gene set forth in SEQ ID NO: 11.

Yet another aspect of the present invention provides a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

Yet another aspect of the present invention provides a composition for producing hydroxy fatty acids, including the recombinant microorganisms including the lipoxygenase gene and the decarboxylase gene.

Yet another aspect of the present invention provides a method for preparing recombinant microorganisms for producing hydroxy fatty acids or secondary fatty alcohols, including introducing a lipoxygenase gene represented by a nucleotide sequence set forth in SEQ ID NOs: 8 to 10 and a decarboxylase gene represented by a nucleotide sequence set forth in SEQ ID NO: 11 into microorganisms.

### Advantageous Effects

It was confirmed that when using lipoxygenase-based recombinant microorganisms according to the present invention as whole-cell biocatalysts, hydroxy fatty acids can be produced from unsaturated fatty acids. In addition, it was confirmed that when using the recombinant microorganisms co-expressing lipoxygenase and Chlorella-derived decarboxylase as whole-cell biocatalysts, secondary fatty alcohols can be produced from unsaturated fatty acids. Thus, the recombinant microorganisms constructed in the present invention can be used in various ways in the field of hydroxy fatty acid and secondary fatty alcohol production.

### DESCRIPTION OF DRAWINGS

FIG. 1 illustrates a pathway transformed into 13-hydroxyoctadecenoic acid (3) through 13-hydroperoxyoctadecenoic acid (2) through a dioxygenation reaction and a reduction reaction of Pseudomonas-derived lipoxygenase from linoleic acid (1). Thereafter, FIG. 1 is a diagram illustrating a biotransformation reaction pathway of producing 6-hydroxyheptadecene (4) by a Chlorella-derived decarboxylase reaction.

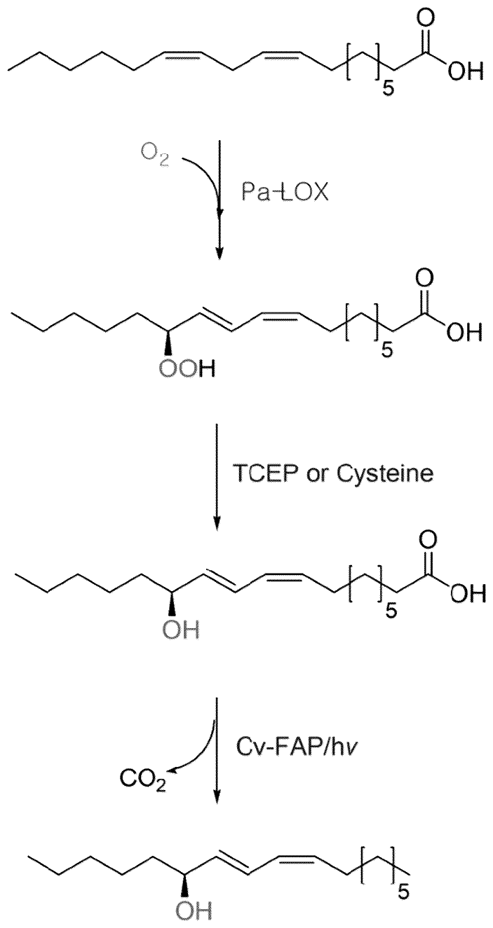

FIG. 2 is a diagram illustrating a biotransformation result using recombinant E. coli pET22b-Pa-LOX according to the present invention when the concentration of linoleic acid is 10 mM.

FIG. 3 is a diagram illustrating a biotransformation result using recombinant E. coli pET22b-Pa-LOX according to the present invention when the concentration of linoleic acid is 100 mM.

FIG. 4 is a diagram illustrating a biotransformation result using recombinant E. coli pET22b-Pa-LOX according to the present invention when the concentration of linoleic acid is 200 mM.

FIG. 5 is a diagram illustrating a biotransformation result using recombinant E. coli pET22b-Pa-LOX according to the present invention when the concentration of linolenic acid is 10 mM.

FIG. 6 is a diagram illustrating a biotransformation result using recombinant E. coli pET22b-Pa-LOX according to the present invention when the concentration of arachidonic acid is 10 mM.

FIG. 7 is a diagram illustrating a biotransformation result using recombinant E. coli pACYC-Pa-LOX/pET28a-Cv-FAP according to the present invention when the concentration of linoleic acid is 12 mM.

FIG. 8 is a diagram illustrating a biotransformation result using recombinant E. coli pET21a-EsLOX according to the present invention when the concentration of linoleic acid is 12 mM.

FIG. 9 is a diagram illustrating a biotransformation result using recombinant E. coli pET21a-EsLOX according to the present invention when the concentration of α-linolenic acid is 10 mM.

FIG. 10 is a diagram illustrating a result of confirming a water-soluble expression level in E. coli and a secretion amount into a medium of an MBP fusion enzyme through SDS-PAGE.

FIG. 11 is a diagram illustrating a biotransformation result using recombinant E. coli pB4-EsLOX expressing an MBP fusion enzyme according to the present invention when the concentration of linoleic acid is 10 mM.

FIG. 12 is a diagram illustrating a biotransformation result using recombinant E. coli pB4-EsLOX expressing an MBP fusion enzyme according to the present invention when the concentration of linoleic acid is 100 mM.

FIG. 13 is a diagram illustrating a biotransformation result using recombinant E. coli pACYC-EsLOX/pET28a-Cv-FAP expressing lipoxygenase and decarboxylase according to the present invention when the concentration of linoleic acid is 10 mM.

FIG. 14 is a diagram illustrating a result of analyzing the purity by GC/MS after purifying a sample by silica gel column chromatography.

FIG. 15 is a diagram illustrating a result of analyzing a sample through NMR.

### BEST MODE FOR THE INVENTION

Hereinafter, the present invention will be described in detail.

### Pseudomonas-Derived Lipoxygenase-Based Recombinant E. coli and Method for Preparing Hydroxy Fatty Acids and Secondary Fatty Alcohols Using the Same

According to an aspect of the present invention, the present invention provides recombinant microorganisms including a lipoxygenase gene represented by a nucleotide sequence set forth in SEQ ID NO: 1 or 2.

In the present invention, the “lipoxygenase” means an enzyme that generates peroxide by adding oxygen to unsaturated fatty acids. The lipoxygenase is also present in microorganisms and animal tissues. In animals, there are 5, 12, and 15-lipoxygenases depending on a difference in position where oxygen is added to arachidonic acid, and the lipoxygenases are contained in bone marrow-derived cells such as platelets and leukocytes, epithelial cells, and nerve cells. The 5-lipoxygenase is involved in the pathophysiology of inflammation or immunity by synthesizing leukotrienes that act on leukocytotaxis or smooth muscle contraction from arachidonic acid via 5-hydroperoxy acid.

In an embodiment of the present invention, the lipoxygenase gene is preferably derived from Pseudomonas aeruginosa.

In an embodiment of the present invention, the lipoxygenase is preferably secreted in the periplasm or cytoplasm.

In the present invention, the “periplasm” is a space between the inner and outer membranes of Gram-negative bacteria, and exhibits a gel-like viscosity due to a very high protein concentration.

In a preferred embodiment of the present invention, the recombinant microorganisms including the lipoxygenase gene include a lipoxygenase gene represented by a nucleotide sequence set forth in SEQ ID NO: 1, and the lipoxygenase may be secreted into the periplasm.

In an embodiment of the present invention, the recombinant microorganisms including the lipoxygenase gene are preferably for producing hydroxy fatty acids or hydroperoxy fatty acids.

In the present invention, the “hydroxy fatty acid” is a form of having one or more hydroxyl groups in a central chain of a general fatty acid, and is more soluble in water than the corresponding fatty acid or alcohol. The hydroxy fatty acids are classified into α-, β-, γ-, and δ-hydroxy fatty acids according to a position of a hydroxyl group.

In a preferred embodiment of the present invention, the hydroxy fatty acids may be at least one selected from the group consisting of 13-hydroxyoctadecadienoic acid, 13-hydroxyoctadecatrienoic acid, and 15-hydroxyeicosatetraenoic acid, but are not limited thereto.

In an embodiment of the present invention, the recombinant microorganisms are preferably for whole-cell biotransformation.

In the present invention, the “biotransformation” means a process of transforming an added substance into a chemically modified form using a physiological function of a living organism. The biotransformation may use enzymes or microorganisms expressing the enzymes.

In the present invention, the “whole-cell biotransformation” refers to a process of transforming an initial substance into a desired substance using a transformant itself (i.e., a whole cell) expressing the enzyme.

According to another aspect of the present invention, the present invention provides recombinant microorganisms, including a lipoxygenase gene represented by a nucleotide sequence set forth in SEQ ID NO: 1 or 2; and a decarboxylase gene represented by a gene set forth in SEQ ID NO: 3.

In an embodiment of the present invention, the lipoxygenase gene and the decarboxylase gene may be introduced into microorganisms simultaneously or separately, and the decarboxylase gene may be introduced into the recombinant microorganisms including the lipoxygenase gene of the above-described aspect of the present invention.

In an embodiment of the present invention, the decarboxylase is preferably derived from Chlorella variabilis.

In an embodiment of the present invention, the recombinant microorganisms including the lipoxygenase gene and the decarboxylase gene include a lipoxygenase gene represented by a nucleotide sequence set forth in SEQ ID NO: 2, and the lipoxygenase may be secreted into the cytoplasm.

In an embodiment of the present invention, the recombinant microorganisms including the lipoxygenase gene and the decarboxylase gene are preferably for producing hydroxy fatty acids, hydroperoxy fatty acids, or secondary fatty alcohols. The recombinant microorganisms including the lipoxygenase gene and the decarboxylase gene may biotransform unsaturated fatty acids to hydroxy fatty acids using the lipoxygenase, and biotransform hydroxy fatty acids to secondary fatty alcohols using the decarboxylase.

In an embodiment of the present invention, the secondary fatty alcohols may be at least one selected from the group consisting of linoleic acid-derived fatty alcohol 6-hydroxy-7,9-heptadecene, γ-linolenic acid-derived fatty alcohol 6-hydroxy-7,9,12-heptadecatriene, α-linolenic acid-derived fatty alcohol 6-hydroxy-3,7,9-heptadecatriene, and oleic acid-derived fatty alcohol 9-hydroxy-8-heptadecene.

According to another aspect of the present invention, the present invention provides a method for producing hydroxy fatty acids, including preparing hydroperoxy fatty acids by reacting unsaturated fatty acids with the recombinant microorganisms including the lipoxygenase gene. In addition, the present invention provides a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

In an embodiment of the present invention, the method may further include reducing the hydroperoxy fatty acids.

In a preferred embodiment of the present invention, the method may further include irradiating light with a wavelength of 400 to 500 nm when producing secondary fatty alcohols using the lipoxygenase gene and the decarboxylase. This step is to induce a light-dependent decarboxylation reaction of decarboxylase. More preferably, light having a wavelength of 450 nm may be irradiated for the light-dependent decarboxylation reaction.

As used herein, the term “unsaturated fatty acids” refer to fatty acids in which a double bond between carbons exists as a chain-shaped compound having a carbon-carbon unsaturated bond and a carboxy group in one molecule. Fatty acid having one double bond is called monoenoic acid, and dienoic acid, trienoic acid, tetraenoic acid, pentaenoic acid, and hexaenoic acid exist in nature. Dienoic acids or higher are collectively referred to as polyenoic acids, and acids higher than tetraenoic acids, which are abundant in fish oil, are called perunsaturated fatty acids. The position of the double bond is indicated by what number of carbons is attached from the carboxyl group, but fatty acids existing in nature have a predetermined arrangement.

In an embodiment of the present invention, the unsaturated fatty acids may be at least one selected from the group consisting of linoleic acid, α-linolenic acid, γ-linolenic acid, arachidonic acid, oleic acid and palmitoleic acid, but are not limited thereto.

In an embodiment of the present invention, the hydroxy fatty acids may be at least one selected from the group consisting of 13-hydroxyoctadecadienoic acid, 13-hydroxyoctadecatrienoic acid, and 15-hydroxyeicosatetraenoic acid, but are not limited thereto.

In an embodiment of the present invention, the secondary fatty alcohols may be at least one selected from the group consisting of linoleic acid-derived fatty alcohol 6-hydroxy-7,9-heptadecene, γ-linolenic acid-derived fatty alcohol 6-hydroxy-7,9,12-heptadecatriene, α-linolenic acid-derived fatty alcohol 6-hydroxy-3,7,9-heptadecatriene, and oleic acid-derived fatty alcohol 9-hydroxy-8-heptadecene.

In an embodiment of the present invention, the media and other culture conditions may be used with any medium used for general culturing of microorganisms. Preferably, the recombinant microorganisms of the present invention are cultured in a conventional medium containing appropriate carbon sources, nitrogen sources, amino acids, vitamins, etc. under aerobic conditions while controlling temperature, pH, and the like.

The medium may include sugars or sugar alcohols as a carbon source, more specifically may include at least one selected from the group consisting of glucose, mannitol, sucrose, arabinose, galactose, glycerol, xylose, mannose, fructose, lactose, maltose, sucrose, alginic acid, cellulose, dextrin, glycogen, hyaluronic acid, lentinan, zymosan, chitosan, glucan, lignin and pectin, preferably may include at least one selected from the group consisting of glucose, mannitol, alginic acid, sucrose, arabinose, galactose and glycerol, but is not limited thereto. As the inorganic compound, sodium chloride, calcium chloride, iron chloride, magnesium sulfate, iron sulfate, manganese sulfate, calcium carbonate, and the like may be used, and in addition, amino acids, vitamins, appropriate precursors, and the like may be included. These media or precursors may be added to a culture medium in a batch or continuous manner.

During the culture, a compound such as ammonium hydroxide, potassium hydroxide, ammonia, phosphoric acid, and sulfuric acid is added to the culture medium by a proper method to adjust the pH of the culture medium. In addition, during the culture, generation of bubbles may be inhibited by using an anti-foaming agent such as fatty acid polyglycol ester. Further, in order to maintain an aerobic state of the culture medium, oxygen or oxygen-containing gases may be injected into the culture medium, or in order to maintain anaerobic and aerobic states, gases are not injected or nitrogen, hydrogen, or carbon dioxide gas may be injected.

The temperature of the culture medium may be set usually 27° C. to 37° C., preferably 30° C. to 35° C. The culturing period may be continued until a desired production amount of a useful substance is obtained, preferably for 10 to 100 hours.

The method according to the present invention may further include additionally purifying or recovering hydroxy fatty acids or secondary fatty alcohols produced in the culturing step. The method for recovering hydroxy fatty acids or secondary fatty alcohols from the recombinant microorganisms or culture medium may be used with methods known in the art, such as centrifugation, filtration, anion exchange chromatography, crystallization, HPLC, etc., but is not limited thereto. The recovering step may include a purification process, and those skilled in the art may select and utilize several well-known purification processes as needed.

According to yet another aspect of the present invention, the present invention provides a composition for producing hydroxy fatty acids, including the recombinant microorganisms including the lipoxygenase gene. In addition, the present invention provides a composition for producing hydroxy fatty acids or secondary fatty alcohols, including the recombinant microorganisms including the lipoxygenase gene and the decarboxylase gene.

The recombinant microorganisms according to the present invention can be used as whole-cell biocatalysts, and can be used as a composition for producing hydroxy fatty acids or secondary fatty alcohols.

In an embodiment of the present invention, the composition may further include a known active ingredient for inducing production of hydroxy fatty acids or secondary fatty alcohols (i.e., inducing whole-cell biotransformation) or culturing recombinant microorganisms.

According to yet another aspect of the present invention, the present invention provides a method for preparing recombinant microorganisms for producing hydroxy fatty acids, including introducing a lipoxygenase gene represented by a nucleotide sequence set forth in SEQ ID NO: 1 or 2 into microorganisms. In addition, the present invention provides a method for preparing recombinant microorganisms for producing hydroxy fatty acids or secondary fatty alcohols, including introducing a lipoxygenase gene represented by a nucleotide sequence set forth in SEQ ID NO: 1 or 2 and a decarboxylase gene represented by a nucleotide sequence set forth in SEQ ID NO: 3 into microorganisms.

In the present invention, the method is not particularly limited to a method for introducing the lipoxygenase gene and the decarboxylase gene into a host gene, but is preferably a method of inserting the gene into a vector and introducing the vector into the recombinant microorganisms using electrophoresis, a heat shock transformation method, etc. to be expressed together in a recombinant strain.

In an embodiment of the present invention, the lipoxygenase gene and the decarboxylase gene may be introduced into the microorganisms simultaneously or separately, or may be introduced sequentially. In addition, the lipoxygenase gene and the decarboxylase gene may be introduced into microorganisms in the form of a single vector including both the genes, and may be introduced into microorganisms in the form of two vectors including each of the two genes, but are not limited thereto.

### Enhygromyxa-Derived Lipoxygenase-Based Recombinant Microorganisms and Method for Preparing Hydroxy Fatty Acids and Secondary Fatty Alcohols Using the Same

According to an aspect of the present invention, the present invention provides recombinant microorganisms including a lipoxygenase gene represented by at least one nucleotide sequence selected from SEQ ID Nos: 8 to 10.

In the present invention, the “lipoxygenase” means an enzyme that generates peroxide by adding oxygen to unsaturated fatty acids. The lipoxygenase is also present in microorganisms and animal tissues. In animals, there are 5, 12, and 15-lipoxygenases depending on a difference in position where oxygen is added to arachidonic acid, and the lipoxygenases are contained in bone marrow-derived cells such as platelets and leukocytes, epithelial cells, and nerve cells. The 5-lipoxygenase is involved in the pathophysiology of inflammation or immunity by synthesizing leukotrienes that act on leukocytotaxis or smooth muscle contraction from arachidonic acid via 5-hydroperoxy acid.

In an embodiment of the present invention, the lipoxygenase gene is preferably derived from Enhygromyxa salina.

In an embodiment of the present invention, the recombinant microorganisms including the lipoxygenase gene preferably includes a lipoxygenase gene represented by a nucleotide sequence set forth in SEQ ID NO: 8.

In an embodiment of the present invention, the recombinant microorganisms preferably further include a maltose binding protein (MBP)-coding sequence represented by a nucleotide sequence set forth in SEQ ID NO: 18.

In an embodiment of the present invention, the lipoxygenase is preferably secreted into the periplasm or cytoplasm.

In the present invention, the “periplasm” is a space between the inner and outer membranes of Gram-negative bacteria, and exhibits a gel-like viscosity due to a very high protein concentration.

In a preferred embodiment of the present invention, the recombinant microorganisms including the lipoxygenase gene include a lipoxygenase gene represented by at least one nucleotide sequence selected from SEQ ID NOs: 8 to 10, and the lipoxygenase may be secreted into the periplasm.

In an embodiment of the present invention, the recombinant microorganisms including the lipoxygenase gene are preferably for producing hydroxy fatty acids or hydroperoxy fatty acids.

In the present invention, the “hydroxy fatty acid” is a form of having one or more hydroxyl groups in a central chain of a general fatty acid, and is more soluble in water than the corresponding fatty acid or alcohol. The hydroxy fatty acids are classified into α-, β-, γ-, and δ-hydroxy fatty acids according to a position of a hydroxyl group.

In a preferred embodiment of the present invention, the hydroxy fatty acids may be at least one selected from the group consisting of 9-hydroxyoctadecadienoic acid and 9-hydroxyoctadecatrienoic acid, but are not limited thereto.

In an embodiment of the present invention, the recombinant microorganisms are preferably for whole-cell biotransformation.

In the present invention, the “biotransformation” means a process of transforming an added substance into a chemically modified form using a physiological function of a living organism. The biotransformation may use enzymes or microorganisms expressing the enzymes.

In the present invention, the “whole-cell biotransformation” refers to a process of transforming an initial substance into a desired substance using a transformant itself (i.e., a whole cell) expressing the enzyme.

In an embodiment of the present invention, the recombinant microorganisms may be characterized to be selected from the group consisting of bacteria, yeast, and fungi, and may preferably Escherichia sp. microorganisms, and more preferably Escherichia coli.

According to another aspect of the present invention, the present invention provides recombinant microorganisms, including a lipoxygenase gene represented by at least one nucleotide sequence selected from SEQ ID NOs: 8 to 10; and a decarboxylase gene represented by a gene set forth in SEQ ID NO: 11.

In an embodiment of the present invention, the lipoxygenase gene and the decarboxylase gene may be introduced into microorganisms simultaneously or separately, and the decarboxylase gene may be introduced into recombinant microorganisms including the lipoxygenase gene of the above-described aspect of the present invention.

In an embodiment of the present invention, the decarboxylase is preferably derived from Chlorella variabilis.

In an embodiment of the present invention, the recombinant microorganisms including the lipoxygenase gene and the decarboxylase gene include a lipoxygenase gene represented by a nucleotide sequence set forth in SEQ ID NO: 8, and the lipoxygenase may be secreted into the cytoplasm.

In an embodiment of the present invention, the recombinant microorganisms including the lipoxygenase gene and the decarboxylase gene are preferably for producing hydroxy fatty acids, hydroperoxy fatty acids, or secondary fatty alcohols. The recombinant microorganisms including the lipoxygenase gene and the decarboxylase gene may biotransform unsaturated fatty acids to hydroxy fatty acids using the lipoxygenase, and biotransform hydroxy fatty acids to secondary fatty alcohols using the decarboxylase.

In an embodiment of the present invention, the secondary fatty alcohols may be at least one selected from the group consisting of linoleic acid-derived fatty alcohol 8-hydroxy-9,11-heptadecene, γ-linolenic acid-derived fatty alcohol 8-hydroxy-5,9,11-heptadecatriene, and α-linolenic acid-derived fatty alcohol 8-hydroxy-9,11,14-heptadecatriene.

According to another aspect of the present invention, the present invention provides a method for producing hydroxy fatty acids, including preparing hydroperoxy fatty acids by reacting unsaturated fatty acids with the recombinant microorganisms including the lipoxygenase gene. In addition, the present invention provides a method for producing hydroxy fatty acids or secondary fatty alcohols, including preparing hydroperoxy fatty acids by reacting unsaturated fatty acids with the recombinant microorganisms including the lipoxygenase gene and the decarboxylase gene.

In an embodiment of the present invention, the method may further include reducing the hydroperoxy fatty acids.

In a preferred embodiment of the present invention, the method may further include irradiating light with a wavelength of 400 to 500 nm when producing secondary fatty alcohols using the lipoxygenase gene and the decarboxylase. This step is to induce a light-dependent decarboxylation of decarboxylase. More preferably, light having a wavelength of 450 nm may be irradiated for the light-dependent decarboxylation.

As used herein, the term “unsaturated fatty acids” refer to fatty acids in which a double bond between carbons exists as a chain-shaped compound having a carbon-carbon unsaturated bond and a carboxy group in one molecule. Fatty acid having one double bond is called monoenoic acid, and dienoic acid, trienoic acid, tetraenoic acid, pentaenoic acid, and hexaenoic acid exist in nature. Dienoic acids or higher are collectively referred to as polyenoic acids, and acids higher than tetraenoic acids, which are abundant in fish oil, are called perunsaturated fatty acids. The position of the double bond is indicated by what number of carbons is attached from the carboxyl group, but fatty acids existing in nature have a predetermined arrangement.

In an embodiment of the present invention, the unsaturated fatty acids may be at least one selected from the group consisting of linoleic acid, α-linolenic acid, γ-linolenic acid, arachidonic acid, oleic acid and palmitoleic acid, but are not limited thereto.

In an embodiment of the present invention, the hydroxy fatty acids may be at least one selected from the group consisting of 9-hydroxyoctadecadienoic acid and 9-hydroxyoctadecatrienoic acid, but are not limited thereto.

In an embodiment of the present invention, the media and other culture conditions may be used with any medium used for general culturing of microorganisms. Preferably, the recombinant microorganisms of the present invention are cultured in a conventional medium containing appropriate carbon sources, nitrogen sources, amino acids, vitamins, etc. under aerobic conditions while controlling temperature, pH, and the like.

The medium may contain sugars or sugar alcohols as a carbon source, more specifically may include at least one selected from the group consisting of glucose, mannitol, sucrose, arabinose, galactose, glycerol, xylose, mannose, fructose, lactose, maltose, sucrose, alginic acid, cellulose, dextrin, glycogen, hyaluronic acid, lentinan, zymosan, chitosan, glucan, lignin and pectin, preferably may include at least one selected from the group consisting of glucose, mannitol, alginic acid, sucrose, arabinose, galactose and glycerol, but is not limited thereto. As the inorganic compound, sodium chloride, calcium chloride, iron chloride, magnesium sulfate, iron sulfate, manganese sulfate, calcium carbonate, and the like may be used, and in addition, amino acids, vitamins, appropriate precursors, and the like may be included. These media or precursors may be added to a culture medium in a batch or continuous manner.

During the culture, a compound such as ammonium hydroxide, potassium hydroxide, ammonia, phosphoric acid, and sulfuric acid is added to the culture medium by a proper method to adjust the pH of the culture medium. In addition, during the culture, generation of bubbles may be inhibited by using an anti-foaming agent such as fatty acid polyglycol ester. Further, in order to maintain an aerobic state of the culture medium, oxygen or oxygen-containing gases may be injected into the culture medium, and in order to maintain anaerobic and aerobic states, gases are not injected or nitrogen, hydrogen, or carbon dioxide gas may be injected.

The temperature of the culture medium may be set usually 27° C. to 37° C., preferably 30° C. to 35° C. The culturing period may be continued until a desired production amount of a useful substance is obtained, preferably for 10 to 100 hours.

The method according to the present invention may further include additionally purifying or recovering hydroxy fatty acids or secondary fatty alcohols produced in the culturing step. The method for recovering hydroxy fatty acids or secondary fatty alcohols from the recombinant microorganisms or culture medium may be used with methods known in the art, such as centrifugation, filtration, anion exchange chromatography, crystallization, HPLC, etc., but is not limited thereto. The recovering step may include a purification process, and those skilled in the art may select and utilize several well-known purification processes as needed.

According to yet another aspect of the present invention, the present invention provides a composition for producing hydroxy fatty acids, including the recombinant microorganisms including the lipoxygenase gene. In addition, the present invention provides a composition for producing hydroxy fatty acids or secondary fatty alcohols, including the recombinant microorganisms including the lipoxygenase gene and the decarboxylase gene.

The recombinant microorganisms according to the present invention can be used as whole-cell biocatalysts, and can be used as a composition for producing hydroxy fatty acids or secondary fatty alcohols.

In an embodiment of the present invention, the composition may further include a known active ingredient for inducing production of hydroxy fatty acids or secondary fatty alcohols (i.e., inducing whole-cell biotransformation) or culturing recombinant microorganisms.

According to yet another aspect of the present invention, the present invention provides a method for preparing recombinant microorganisms for producing hydroxy fatty acids, including introducing a lipoxygenase gene represented by at least one nucleotide sequence selected from SEQ ID NOs: 8 to 10 into microorganisms. In addition, the present invention provides a method for preparing recombinant microorganisms for producing hydroxy fatty acids or secondary fatty alcohols, including introducing a lipoxygenase gene represented by at least one nucleotide sequence selected from SEQ ID NOs: 8 to 10 and a decarboxylase gene represented by a nucleotide sequence set forth in SEQ ID NO: 11 into microorganisms.

In the present invention, the method is not particularly limited to a method for introducing the lipoxygenase gene and the decarboxylase gene into a host gene, but is preferably a method of inserting the gene to a vector and introducing the vector into the recombinant microorganisms using electrophoresis, a heat shock transformation method, etc. to be expressed together in a recombinant strain.

In an embodiment of the present invention, the lipoxygenase gene and the decarboxylase gene may be introduced into the microorganisms simultaneously or separately, or may be introduced sequentially. In addition, the lipoxygenase gene and the decarboxylase gene may be introduced into microorganisms in the form of a single vector including both the genes, and may be introduced into microorganisms in the form of two vectors including each of the two genes, but are not limited thereto.

Duplicated contents are omitted in consideration of the complexity of the present specification, and terms not defined otherwise in the present specification have the meanings commonly used in the art to which the present invention pertains.

### Modes for the Invention

### Pseudomonas Lipoxygenase-Based Recombinant Microorganisms and Method for Preparing Hydroxy Fatty Acids and Secondary Fatty Alcohols Using the Same

### Example 1. Preparation of 13-Hydroperoxyoctadecenoic Acid From Linoleic Acid Using Recombinant E. coli

1-1. Construction of Whole-Cell Catalysts Based on Recombinant Plasmid and E. coli

Vectors pET22b-Pa-LOX (lipoxygenase secreted into the periplasm) and pACYC-Pa-LOX (lipoxygenase secreted into the cytoplasm) including a lipoxygenase gene catalyzing a dioxygenation reaction of the present invention were prepared. A vector pET-22b(+) included an N-terminal PelB signal sequence for secretion into the periplasm and his tag for purification, and a vector pACYC included an N-terminal his tag.

Specifically, a polymerase chain reaction was performed based on genomic DNA of Pseudomonas aeruginosa to amplify a lipoxygenase gene. The lipoxygenase gene for insertion into the vector pET22b-Pa-LOX was amplified with primers 5′-CGGCGATGGCCATGCATCACCATCATCACCAC-3′ (SEQ ID NO: 4) and 3′-GCTCGTGGTTATAGACTTTCGAACGCCGGCG-5′ (SEQ ID NO: 5). The lipoxygenase gene for insertion into the vector pACYC-Pa-LOX was amplified with 5′-GCCAGGATCCGAATTCGAATGACTCGATATTCTTTTCAC-3′ (SEQ ID NO: 6) and 3′-CTCGTGGTTATAGACTTTCGAACGCCGGCGTA-5′ (SEQ ID NO: 7). Lipoxygenase gene sequences for insertion into the vectors pET22b-Pa-LOX and pACYC-Pa-LOX obtained through PCR were represented by SEQ ID NOs: 1 and 2, respectively.

The amplified P. aeruginosa lipoxygenase DNA fragment was purified using a PCR purification kit (QIAGEN, Hilden, Germany). The vector pET22b(+) was cleaved using NcoI and HindIII restriction enzymes, and the vector pACYC was cleaved using EcoRI and HindIII restriction enzymes. The purified PCR product was inserted into the vectors pET-22b(+) and pACYC cleaved with the restriction enzymes, respectively, using an In-Fusion® HD Cloning Kit (Takara, Tokyo, Japan) to prepare the vectors pET22b-Pa-LOX and pACYC-Pa-LOX including the lipoxygenase gene.

The expression vectors pET22b-Pa-LOX and pACYC-Pa-LOX prepared by the method were transformed into E. coli BL21 (DE3), respectively. The recombinant E. coli was grown in LB media containing 100 μg/mL of ampicillin and 30 μg/mL of chloramphenicol, respectively.

### 1-2. Biotransformation According to Concentration of Linoleic Acid

For the dioxygenation reaction of linoleic acid using whole-cell biocatalysts based on the recombinant E. coli pET22b-Pa-LOX prepared in Example 1-1, the recombinant E. coli expressing the lipoxygenase gene was cultured at 37° C. in a terrific medium. After inducing gene expression with IPTG, a biotransformation reaction was performed in a 50 mM potassium phosphate buffer with 3.6 g/L of dried E. coli cells after cultured at 16° C. for 22 hours. The biotransformation was performed at 200 rpm and 30° C. in a flask in a shaking incubator. Specifically, hydroperoxy fatty acid (13-hydroperoxyoctadecadienoic acid (13-HpODE)(3)) was produced by adding linoleic acid at various concentrations of 10, 100, and 200 mM to the culture medium. Then, in order to reduce the hydroperoxy fatty acid, the reaction solution was added with a reducing agent (TCEP) at concentrations twice or more than substrate concentrations of 25, 200, and 200 mM and then reacted for 30 minutes.

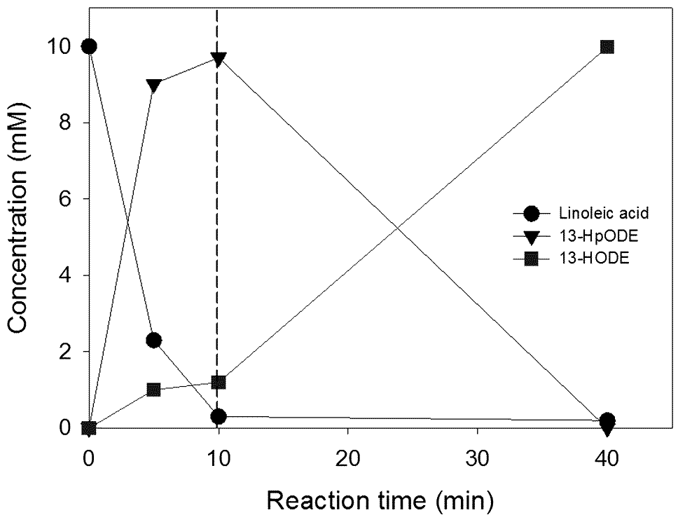

A unit (U), which is a unit of total cell activity, was defined as μmol of hydroperoxy fatty acid produced for 1 minute using 1.0 g of dry cells at 30° C. The biotransformation results according to a concentration of linoleic acid were illustrated in FIGS. 2 to 4, respectively.

As illustrated in FIG. 2, it was confirmed that recombinant E. coli pET22b-Pa-LOX produced hydroxy fatty acid (13-hydroxyoctadecadienoic acid (13-HODE)(3)) when linoleic acid was 10 mM. More specifically, it was confirmed that when linoleic acid was reacted at a concentration of 10 mM, 97% or more of linoleic acid was transformed to hydroperoxy fatty acid 13-HpODE (2) only for 10 minutes. In addition, as a result of reducing hydroperoxy fatty acids, it was confirmed that hydroperoxy fatty acid was transformed to hydroxy fatty acid 13-HODE(3), and 99% or more of hydroperoxy fatty acid was transformed from an initial reactant, linoleic acid.

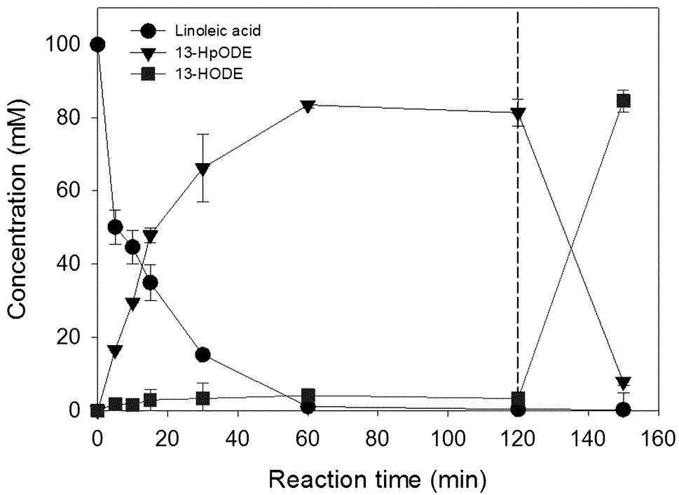

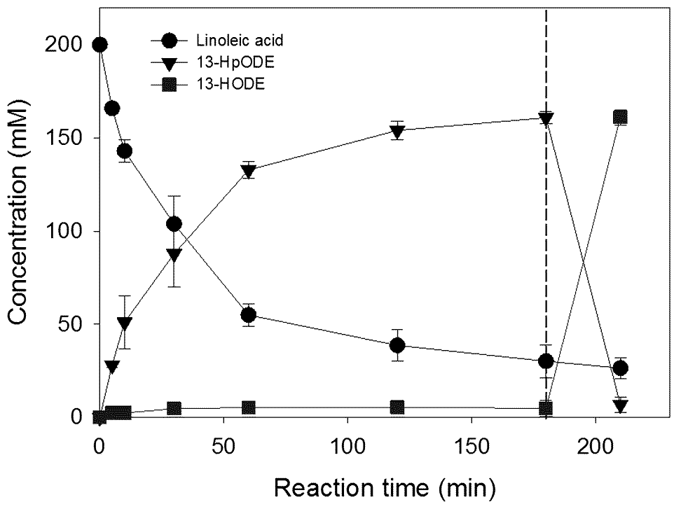

As illustrated in FIGS. 3 and 4, it was confirmed that recombinant E. coli pET22b-Pa-LOX produced hydroxy fatty acid 13-HODE when the concentration of linoleic acid was 100 and 200 mM, respectively. More specifically, when the biotransformation started from 100 mM linoleic acid, the linoleic acid was transformed to 81 mM 13-HpODE only in 2 hours, and added with 200 mM a reducing agent (TCEP) to obtain 85 mM 13-HODE only in 30 minutes (FIG. 3). In addition, it was confirmed when the biotransformation started from 200 mM linoleic acid, the linoleic acid was transformed to 161 mM 13-HpODE only in 3 hours, and reduced to 161 mM 13-HODE at 30 minutes after treated with the reducing agent (FIG. 4). That is, it was confirmed that even when a high concentration of linoleic acid was used, 81% or more of 13-HODE was transformed from linoleic acid, which was the initial reactant. At this time, it was confirmed that about 4 mM hydroperoxy fatty acids were reduced to hydroxy fatty acids within the biotransformed cells without treatment with the reducing agent.

The rate of the initial dioxygenation reaction of the biotransformation reaction using the recombinant E. coli was about 800 μmol/g dry cells/min (800 U/g dry cells) or more even at high-concentration linoleic acid.

The result means that the dioxygenation reaction of the recombinant E. coli-based whole-cell catalyst into which Pseudomonas aeruginosa-derived lipoxygenase was introduced was well performed at a rate of 800 U/g dry cells even for a high-concentration of linoleic acid, and as a result, it was possible to produce 13-hydroperoxyoctadecadienoic acid or 13-hydroxyoctadecadienoic acid from linoleic acid in a high concentration.

A result of recombinant E. coli-based whole-cell catalyst biotransformation using 10, 100, and 200 mM linoleic acids confirmed in Example; and a result of conventional Pseudomonas-based whole-cell catalyst biotransformation using 71 mM oleic acid (Comparative Example 1) were compared. The comparison results were shown in Table 1.

TABLE 1

| | Substrate | Initial | Transfor- | Product |
|---|---|---|---|---|
| | concen- | biotransfor- | mation | concen- |
| | tration | mation rate | rate | tration |
| | (mM) | (U/g dry cells)¹ | (%)² | (mM) |
| Example 1-2 | 10 | 500 | 99 | 10 |
| | 100 | 710 | 85 | 85 |
| | 200 | 890 | 85 | 169 |
| Comparative | 71³ | 12³ | 38 | 27 |
| Example³ | | | | |

As shown in Table 1, it was confirmed that the initial rate of the recombinant E. coli-based biotransformation of whole-cell catalysts was at least 74 times faster than the biotransformation results of Pseudomonas whole-cell biocatalysts using oleic acid as a substrate, and the transformation rate was also at least twice higher from 38% to at least 85%.

Therefore, it was supported that the recombinant E. coli-based whole-cell catalyst biotransformation system constructed in Example 1-1 had better activity than the existing Pseudomonas whole-cell biocatalyst biotransformation system.

### 1-3. Biotransformation of Linolenic Acid

In order to determine whether the E. coli-based whole-cell catalyst prepared in Example 1-1 may be applied even to other unsaturated fatty acids, the dioxygenation reaction of lipoxygenase was also confirmed for linolenic acid (i.e., (9Z,12Z)-9,12-octadecadienoic acid). The chemical structure of linolenic acid was identical to linoleic acid except that the number of double bonds in a carbon backbone was one more than that of linoleic acid. Accordingly, oxygen was introduced into the carbon skeleton at position 13 which was the same position as linoleic acid, to be by lipoxygenase transformed into 13-hydroperoxyoctadecatrienoic acid.

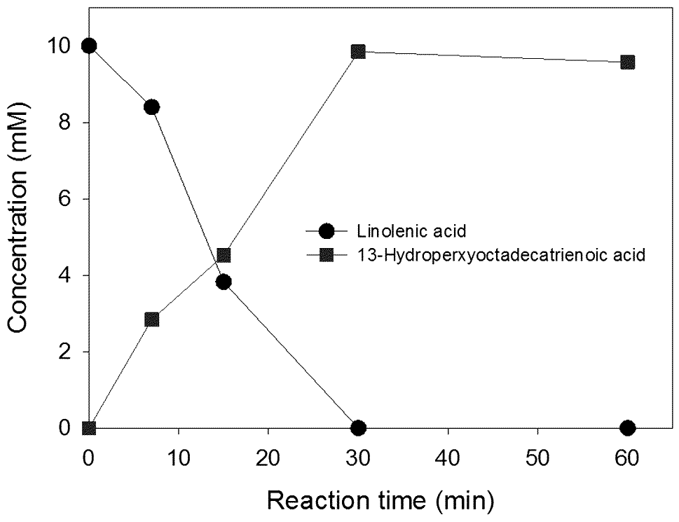

Specifically, the culturing of recombinant E. coli pET22b-Pa-LOX and the expression of lipoxygenase were performed in the same manner as described in Example 1-2, and the biotransformation reaction was performed by adding 10 mM linolenic acid. The results of biotransformation using linolenic acid were shown in FIG. 5.

As illustrated in FIG. 5, it was confirmed that when biotransforming 10 mM linolenic acid using the recombinant E. coli pET22b-Pa-LOX, linolenic acid was all reduced at 1 hour and at least 95% of linolenic acid was transformed to 13-hydroperoxyoctadecatrienoic acid as a product.

The result means that even with respect to not only linoleic acid, but also the other unsaturated fatty acid, linolenic acid, the recombinant E. coli pET22b-Pa-LOX may be applied with the dioxygenation reaction as a biotransformation reaction of the recombinant E. coli-based whole-cell catalyst.

### 1-4. Biotransformation of Arachidonic Acid

In order to expand the application range of a whole-cell catalytic reaction introduced with recombinant E. coli pET22b-Pa-LOX-based lipoxygenase, the dioxygenation reaction was confirmed using arachidonic acid (i.e., (5Z,8Z,11Z,14Z)-5,8,11,14-eicosatetraenoic acid) as a type of other unsaturated fatty acids. The chemical structure of arachidonic acid had 20 carbon skeletons and 4 double bonds, and had two more carbon skeletons and two more double bonds than linoleic acid. Accordingly, in the position where oxygen was introduced by lipoxygenase, in the case of linoleic acid, oxygen was introduced into the 13-th carbon skeleton, but in the case of arachidonic acid, oxygen was introduced into the 15-th carbon backbone, and as a result, the arachidonic acid was transformed to 15-hydroperoxyeicosatetraenoic acid.

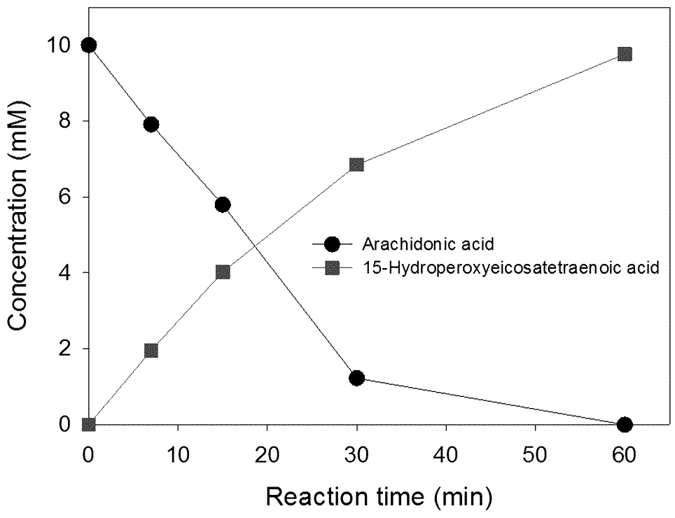

Specifically, the culturing of the recombinant E. coli pET22b-Pa-LOX and the expression of lipoxygenase were performed in the same manner as described in Example 1-2, and the biotransformation reaction was performed by adding 10 mM arachidonic acid. The biotransformation results were illustrated in FIG. 6.

As illustrated in FIG. 6, it was confirmed that in the recombinant E. coli pET22b-Pa-LOX, arachidonic acid was all reduced at 1 hour of the reaction and at least 95% of arachidonic acid was transformed to 15-hydroperoxyeicosatetraenoic acid as a product. The biotransformation rate of arachidonic acid was lower than the biotransformation rate of linoleic acid, which is expected as a rate difference according to a chemical structural difference in unsaturated fatty acids.

The result means that the dioxygenation reaction may be applied as a biotransformation reaction of recombinant E. coli-based whole-cell catalysts even to the arachidonic acid, which is a type of unsaturated fatty acids with different numbers of carbon skeletons and double bonds, in addition to the linoleic acid.

### Example 2. Preparation of 6S-hydroxy-(7E,9Z)-heptadecadiene (6-HHD) From Linoleic Acid Using Recombinant E. coli

2-1. Construction of Whole-Cell Catalysts Based on Recombinant Plasmid and E. coli

In the present invention, in order to prepare secondary fatty alcohol 6-HHD, which was a novel substance, from linoleic acid, recombinant E. coli was constructed by co-introducing a Pseudomonas aeruginosa-derived lipoxygenase gene; and a Chlorella variabilis-derived decarboxylase gene catalyzing a light-dependent decarboxylation reaction.

Specifically, a vector pET28a-Cv-FAP was constructed by introducing a Cv-FAP gene (SEQ ID NO: 3) as decarboxylase catalyzing photodecarboxylation reaction, into a pET28a vector. The vector pACYC-Pa-LOX constructed in Example 1-1; and a vector pET28a-Cv-FAP were transformed to E. coli BL21 (DE3). The constructed recombinant E. coli pACYC-Pa-LOX/pET28a-Cv-FAP was grown in the LB medium containing 50 g/mL of kanamycin and 30 μg/mL of chloramphenicol.

### 2-2. Preparation of 6-HHD From Linoleic Acid

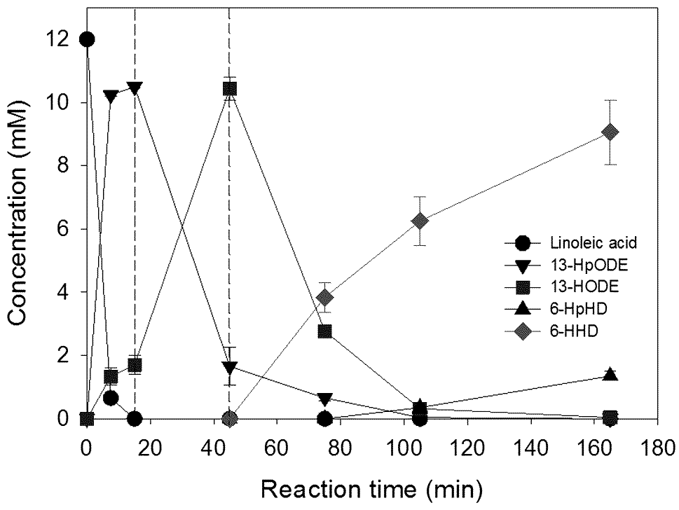

6-HDD was prepared from linoleic acid using whole-cell biocatalysts based on the recombinant E. coli pACYC-Pa-LOX/pET28a-Cv-FAP constructed in Example 2-1. Specifically, the constructed recombinant E. coli pACYC-Pa-LOX/pET28a-Cv-FAP was cultured in a terrific medium at 37° C. After inducing gene expression with IPTG, the recombinant E. coli was cultured at 16° C. for 22 hours. Thereafter, hydroperoxy fatty acids were produced using 12 mM linoleic acid in a 50 mM potassium phosphate buffer with 14.4 g/L of dried E. coli cells. Then, in order to reduce the hydroperoxy fatty acid, the reaction solution was added with a reducing agent (TCEP) at a concentration (25 mM) twice or more than the substrate and then reacted for 30 minutes. Thereafter, the whole-cell catalytic reaction was performed under blue LED light with a 450 nm wavelength band for light-dependent decarboxylation reaction. The biotransformation reaction was performed at 200 rpm and 30° C. in a flask in a shaking incubator, and the decarboxylation reaction was performed at 37° C. in a heating mantle. The biotransformation results were illustrated in FIG. 7.

As illustrated in FIG. 7, it was confirmed that in the recombinant E. coli pACYC-Pa-LOX/pET28a-Cv-FAP, at least 92% of linoleic acid was transformed to 13-hydroperoxyoctadecadienoic acid (13-HpODE) only in 15 minutes in the first step of 6-HHD production. Thereafter, as a result of reduction by adding a reducing agent (second step), it was confirmed that 13-hydroperoxyoctadecadienoic acid (2) was transformed to 10.5 mM 13-hydroxyoctadecadienoic acid (3). As a result of the light-dependent decarboxylation reaction, 9.1 mM 6-HHD (4) as secondary fatty alcohol was obtained from 12 mM linoleic acid. That is, it was confirmed that it took 2 hours and 45 minutes to produce 6-HHD from linoleic acid, and the transformation rate was 76%. At this time, it was confirmed that 6-hydroperoxyheptadecene (6-HpHD) was produced as a by-product by a decarboxylation reaction from 13-hydroperoxyoctadecadienoic acid which has remained, not reduced after the second reaction.

Accordingly, the result means that an E. coli-based whole-cell catalyst co-expressing a Pseudomonas aeruginosa-derived lipoxygenase catalyzing the dioxidation reaction; and a Chlorella variabilis-derived decarboxylase catalyzing light-dependent decarboxylation reaction may produce 6-hydroxyheptadecene (6-HHD) as secondary fatty alcohol from linoleic acid.

In summary, the present inventors constructed recombinant E. coil including a Pseudomonas aeruginosa-derived lipoxygenase and confirmed that when the constructed recombinant E. coil was used as a whole-cell biocatalyst, hydroxy fatty acids may be produced from unsaturated fatty acids such as linoleic acid. In addition, the present inventors constructed a recombinant strain co-expressing the Pseudomonas aeruginosa-derived lipoxygenase and the Chlorella variabilis-derived decarboxylase, and confirmed that when the recombinant strain was used as a whole-cell biocatalyst, secondary fatty alcohols may be produced from unsaturated fatty acids such as linoleic acid. The recombinant E. coli constructed in the present invention can be used in various ways in the production field of hydroxy fatty acids and secondary fatty alcohols.

### Enhygromyxa-Derived Lipoxygenase-Based Recombinant Microorganisms and Method for Preparing Hydroxy Fatty Acids and Secondary Fatty Alcohols Using the Same

### Example 1. Reaction Characteristics of Enhygromyxa salina-Derived Lipoxygenase (EsLOX)

### 1-1. Preparation of Enzyme

A polymerase chain reaction was performed based on genomic DNA of Enhygromyxa salina to amplify a lipoxygenase gene. The lipoxygenase gene for insertion into the vector pET21a-EsLOX was amplified with primers 5′-CGGCGATGGCCATGCATCACCATCATCACCAC-3′ (SEQ ID NO: 12) and 3′-GCTCGTGGTTATAGACTTTCGAACGCCGGCG-5′ (SEQ ID NO: 13). The lipoxygenase gene for insertion into the vector pACYC-EsLOX was amplified with 5′-GGAGATATACCATGGATGAAATACCTGCTGCC-3′ (SEQ ID NO: 14) and 5′-CGGCCGCAAGCTTTCAGATGTTGATG-3′ (SEQ ID NO: 15). Lipoxygenase gene sequences obtained through PCR were represented by SEQ ID NOs: 8 (pET) and 9 (pACYC), respectively.

The amplified Enhygromyxa salina lipoxygenase DNA fragment was purified using a PCR purification kit (QIAGEN, Hilden, Germany). The vector pET21a(+) was cleaved using NcoI and HindIII restriction enzymes, and the vector pACYC was cleaved using NcoI and HindIII restriction enzymes. The purified PCR product was inserted into the vectors pET21a(+), pACYC, and pB4 cleaved with the restriction enzymes, respectively, using an In-Fusion® HD Cloning Kit (Takara, Tokyo, Japan) to prepare the vectors pET21a-EsLOX and pACYC-EsLOX including the lipoxygenase gene.

The expression vectors ppET21a-EsLOX and pACYC-EsLOX prepared by the method were transformed into E. coli BL21 (DE3), respectively. The recombinant E. coli was grown in LB media containing 100 μg/mL of ampicillin and 30 μg/mL of chloramphenicol, respectively.

Thereafter, in order to express the lipoxygenase gene in the recombinant E. coli pET21a-EsLOX, the recombinant E. coli was cultured at 37° C. in a terrific medium, and added with IPTG in the medium at an OD 0.6 to induce the gene expression, and then cultured at 16° C. for 22 hours.

The culture medium was centrifuged at 13,000×g at 4° C. for 30 minutes and washed twice with phosphate-buffered saline, and added with 50 mM monosodium phosphate (NaH₂PO₄), 300 mM sodium chloride, 10 mM imidazole, 0.1 mM protease inhibitor (phenylmethylsulfonyl fluoride), and 400 U lysozyme, and then the cell solution was lysed with a sonicator. The cell lysate was centrifuged at 13,000×g at 4° C. for 10 minutes, and then the pellet was removed to separate only a cell supernatant. The cell supernatant was flowed onto a resin packed with an Ni-NTA agarose resin (Qiagen). The protein bound to the resin was washed with a wash buffer (50 mM NaH₂PO₄, 300 mM NaCl, 20/30 mM imidazole), eluted with an elution buffer (50 mM NaH₂PO₄, 300 mM NaCl, 250 mM imidazole) to purify Enhygromyxa lipoxygenase. Thereafter, a sample containing the purified Enhygromyxa lipoxygenase was prepared by obtaining a concentrated protein solution using 30 kDa Amicon.

### 1-2. Enzymatic Activity for Various Long-Chain Unsaturated Fatty Acids

Then, the enzymatic activity of the enzyme sample was measured using a UV-vis spectrophotometer (234 nm). More specifically, as the linoleic acid dioxygenation reaction progressed, the rate of increase in absorbance of a product, conjugated diene, was measured using the spectrophotometer, and the reaction time was measured for each of 10 seconds up to 180 seconds. The enzymatic activity was measured for unsaturated fatty acids such as linoleic acid, α-linolenic acid, γ-linolenic acid, and arachidonic acid in 50 mM EPPS (pH 8.5) buffer.

As a result of measuring the activity at 234 nm using linoleic acid as a substrate, a K_{M} (mM) value was measured as 0.09 and a k_{cat}/K_{M} (s⁻¹ μM⁻¹) value was measured as 1.51. As a result of measuring the activity at 234 nm using α-linolenic acid as a substrate, a K_{M} (mM) value was measured as 0.12 and a k_{cat}/K_{M} (s⁻¹ μM⁻¹) value was measured as 0.56. As a result of measuring the activity at 234 nm using γ-linolenic acid as a substrate, a K_{M} (mM) value was measured as 0.13 and a k_{cat}/K_{M} (s⁻¹ μM⁻¹) value was measured as 0.61. As a result of measuring the activity at 234 nm using arachidonic acid as a substrate, a K_{M} (mM) value was measured as 0.08 and a k_{cat}/K_{M} (s⁻¹ μM⁻¹) value was measured as 0.29.

The result means that Enhygromyxa salina-derived lipoxygenase has dioxygenation reaction activity for various unsaturated fatty acids, and particularly, has high activity for linoleic acid. This means that 9-hydroxy fatty acid or 9-hydroperoxy fatty acid may be produced from various unsaturated fatty acids.

Table 2 showed a result of measuring the enzymatic activity using various unsaturated fatty acids confirmed in Example.

TABLE 2

| | | | k_{cat}/K_{M} |
|---|---|---|---|
| | Substrate | K_{M} (mM) | (s⁻¹ μM⁻¹) |
| Example 1-2 | Linoleic acid | 0.09 | 1.51 |
| | α-Linolenic acid | 0.12 | 0.56 |
| | γ-Linolenic acid | 0.13 | 0.61 |
| | Arachidonic acid | 0.08 | 0.29 |

### Example 2. Preparation of 9-Hydroperoxyoctadecadienoic Acid From Linoleic Acid Using Recombinant E. coli

### 2-1. Biotransformation of Linoleic Acid

For the dioxygenation reaction of linoleic acid using whole-cell biocatalysts based on the recombinant E. coli pET21a-EsLOX prepared in Example 1-1, the recombinant E. coli expressing the lipoxygenase gene was cultured at 37° C. in a terrific medium. After inducing gene expression with IPTG, a biotransformation reaction was performed in a 50 mM EPPS buffer with 3.6 g/L of dried E. coli cells after cultured at 16° C. for 22 hours. The biotransformation was performed under conditions of 200 rpm and 25° C. in a flask in a shaking incubator. Specifically, hydroperoxy fatty acid (9-hydroperoxyoctadecadienoic acid (9-HpODE)(3)) was produced by adding linoleic acid at various concentrations of 12 and 20 mM to the culture medium. Then, in order to reduce the hydroperoxy fatty acid, the reaction solution was added with a reducing agent (TCEP) at a concentration twice or more than substrate concentrations of 12 and 20 mM and then reacted for 20 minutes.

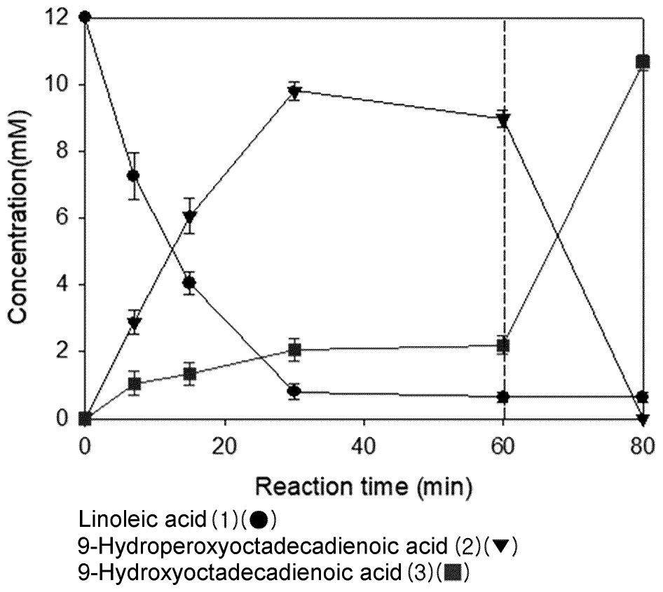

A unit (U), which was a unit of total cell activity, was defined as μmol of hydroperoxy fatty acid produced for 1 minute using 1.0 g of dry cells at 25° C. The results of biotransformation according to a concentration of linoleic acid were shown in FIG. 8.

As illustrated in FIG. 8, it was confirmed that in the recombinant E. coli pET21a-EsLOX produced hydroxy fatty acid (9-hydroxyoctadecadienoic acid (9-HODE)(3)) in the case of 12 mM linoleic acid. More specifically, it was confirmed that 80% or more was transformed to hydroxy fatty acid 9-HpODE(2) only in 30 minutes when linoleic acid was reacted at a concentration of 12 mM. In addition, as a result of reducing the hydroperoxy fatty acid, it was confirmed that the hydroperoxy fatty acid was transformed to hydroxy fatty acid 9-HODE(3), and 89% or more of hydroperoxy fatty acid was transformed from an initial reactant, linoleic acid.

### 2-2. Biotransformation of Linolenic Acid

In order to determine whether the E. coli-based whole-cell catalyst prepared in Example 2-1 may be applied even to other unsaturated fatty acids, the dioxygenation reaction of lipoxygenase was also confirmed for linolenic acid (i.e., (9Z,12Z)-9,12-octadecadienoic acid). The chemical structure of linolenic acid was identical to linoleic acid except that the number of double bonds in a carbon backbone was one more than that of linoleic acid. Accordingly, oxygen was introduced into the carbon skeleton at position 9 which was the same position as linoleic acid, by lipoxygenase to be transformed into 9-hydroperoxyoctadecatrienoic acid.

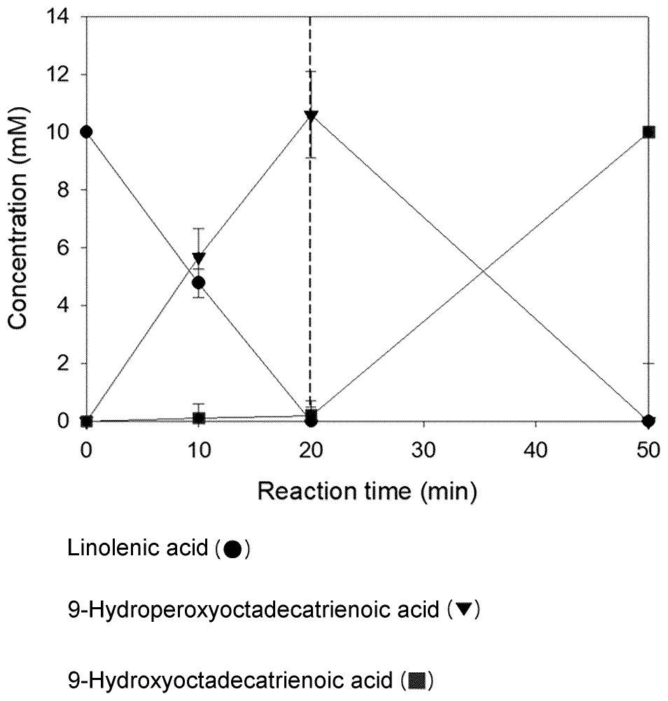

Specifically, the culturing of the recombinant E. coli pET21a-EsLOX and the expression of lipoxygenase were performed in the same manner as described in Example 2-1, and the biotransformation reaction was performed by adding 10 mM linolenic acid. The results of biotransformation using linolenic acid were shown in FIG. 9.

As illustrated in FIG. 9, it was confirmed that when biotransforming 10 mM linolenic acid using the recombinant E. coli pET21a-EsLOX, the linolenic acid was all reduced only in 20 minutes, and at least 90% of linolenic acid was transformed to the product, 9-hydroperoxyoctadecatrienoic acid.

The result means that even with respect to not only linoleic acid, but also the other unsaturated fatty acid, linolenic acid, the recombinant E. coli pET21a-EsLOX may be applied with the dioxygenation reaction as a biotransformation reaction of the recombinant E. coli-based whole-cell catalyst.

### Example 3. Construction of MBP-EsLOX Fusion Enzyme for Improvement of Water-Soluble Expression of EsLOX in E. coli

3-1. Construction of Whole-Cell Catalysts Based on Recombinant Plasmid and E. coli

A vector pB4-EsLOX was constructed by subcloning the lipoxygenase gene of the present invention catalyzing the dioxygenation reaction into a pB4 E. coli expression vector containing a maltose binding protein (MBP)-coding sequence. A vector pB4 included an N-terminal his tag for purification and included a maltose binding protein (MBP)-coding sequence (SEQ ID NO: 18).

Specifically, a polymerase chain reaction was performed to amplify a lipoxygenase gene. The lipoxygenase gene for insertion into the vector pB4-EsLOX was amplified with primers 5′-GCGGTGGTGGCGGCATGTCCAACATCCAA-3′ (SEQ ID NO: 16) and 5′-GCCCTCAGATGTTGATGCTCAGATGTTGAT-3′ (SEQ ID NO: 17). A lipoxygenase gene sequence for insertion into the vector pB4-EsLOX obtained through PCR was represented by SEQ ID NO: 10.

The amplified Enhygromyxa salina lipoxygenase DNA fragment was purified using a PCR purification kit (QIAGEN, Hilden, Germany). The vector pB4 was cleaved using an SmaI restriction enzyme. The purified PCR product was inserted into the vector pB4 cleaved with the restriction enzymes, respectively, using an In-Fusion® HD Cloning Kit (Takara, Tokyo, Japan) to prepare the vector pB4-EsLOX including the lipoxygenase gene.

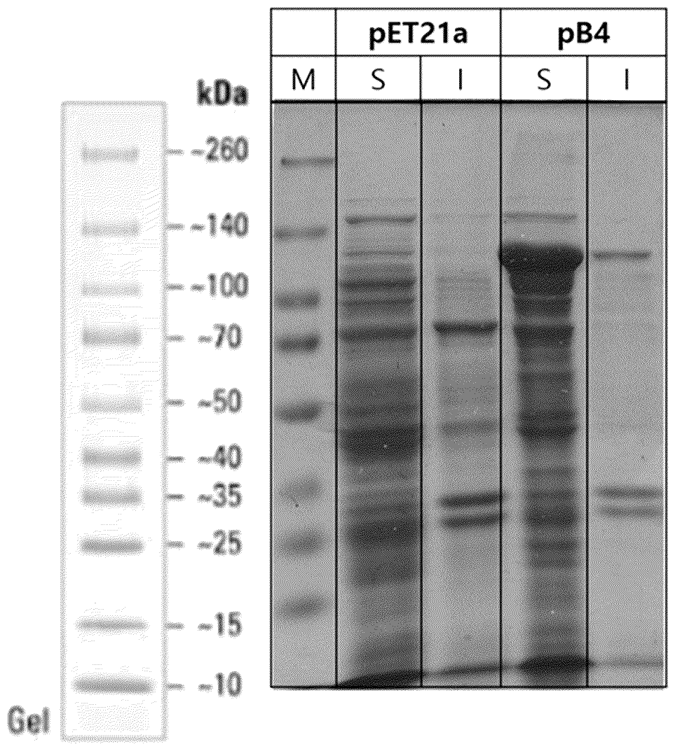

The expression vector pB4-EsLOX constructed by the method was transformed into E. coli BL21 (DE3) star. The recombinant E. coli was grown in an LB medium containing 100 μg/mL of ampicillin. The water-soluble expression level in E. coli and the amount of secretion into the medium of the MBP fusion enzyme were confirmed through SDS-PAGE and a gel analyzer program, and the results were illustrated in FIG. 10.

As illustrated in FIG. 10, it was confirmed that 71 kDa of EsLOX was fused with 44 kDa of MBP to be produced in a size of 115 kDa. In addition, as the analysis result of the secretion amount, it was confirmed that the water-soluble protein of EsLOX increased by about 2 times or more.

### 3-2. Biotransformation According to Concentration of Linoleic Acid

For the dioxygenation reaction of linoleic acid using whole-cell biocatalysts based on the recombinant E. coli pB4-EsLOX prepared in Example 3-1, the recombinant E. coli expressing the lipoxygenase gene was cultured at 37° C. in a terrific medium. After inducing gene expression with IPTG, a biotransformation reaction was performed in a 50 mM EPPS buffer with 3.6 g/L of dried E. coli cells after cultured at 16° C. for 22 hours. The biotransformation was performed under conditions of 200 rpm and 25° C. in a flask in a shaking incubator. Specifically, hydroperoxy fatty acid (9-hydroperoxyoctadecadienoic acid (9-HpODE)(3)) was produced by adding linoleic acid (10 mM) to the culture medium. Thereafter, in order to reduce the hydroperoxy fatty acid, the reaction solution was added with a reducing agent (TCEP) at a concentration twice or more than substrate concentration (10 mM) and then reacted for 20 minutes.

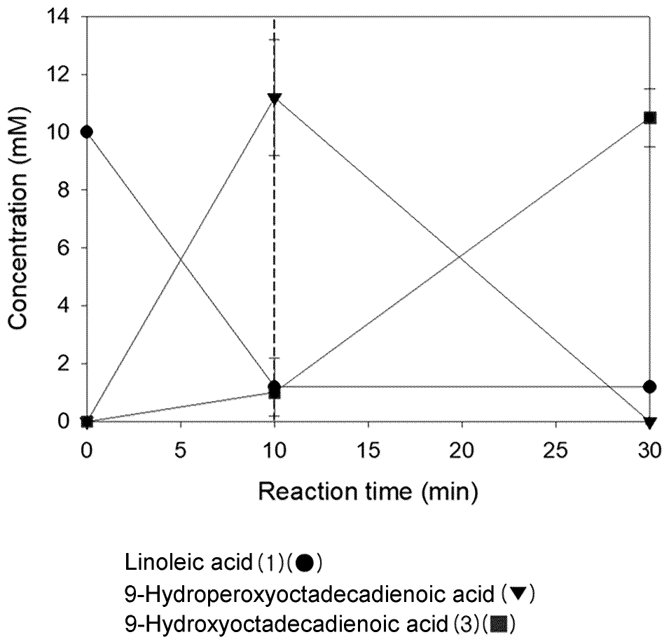

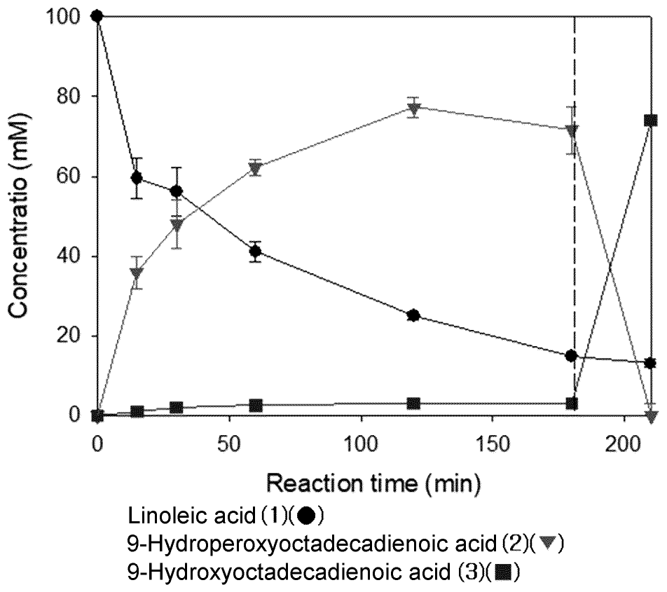

A unit (U), which was a unit of total cell activity, was defined as μmol of hydroperoxy fatty acid produced for 1 minute using 1.0 g of dry cells at 25° C. Biotransformation results according to linoleic acid concentrations of 10 and 100 mM were illustrated in FIGS. 11 and 12.

As illustrated in FIG. 11, it was confirmed that the recombinant E. coli pB4-EsLOX produced hydroxy fatty acid (9-hydroxyoctadecadienoic acid (9-HODE)(3)) in the case of 10 mM linoleic acid. More specifically, it was confirmed that when linoleic acid was reacted at a concentration of 10 mM, 90% or more of linoleic acid was transformed to hydroperoxy fatty acid 9-HpODE(2) only in 10 minutes. In addition, as a result of reducing the hydroperoxy fatty acid, it was confirmed that the hydroperoxy fatty acid was transformed to hydroxy fatty acid 9-HODE(3), and 91% or more of hydroperoxy fatty acid was transformed from an initial reactant, linoleic acid.

As illustrated in FIG. 12, it was confirmed that the recombinant E. coli pB4-EsLOX produced hydroxy fatty acid 9-HODE(3) even when the concentration of linoleic acid was 100 mM. More specifically, when the biotransformation started from 100 mM linoleic acid, the linoleic acid was transformed to 79 mM 9-HpODE(2) only in 2 hours, and added with 200 mM a reducing agent (TCEP) to obtain 80 mM 9-HODE(3) only in 20 minutes. It was confirmed that 80% or more was transformed from linoleic acid, the first reactant. At this time, it was confirmed that about 1 mM hydroperoxy fatty acids were reduced by themselves to hydroxy fatty acids within the biotransformed cells without treatment with the reducing agent.

The rate of the initial dioxygenation reaction of the biotransformation reaction using the recombinant E. coli was about 665 μmol/g dry cells/min (170 U/g dry cells) or more even at high-concentration linoleic acid.

The results showed that improved water-soluble expression of the target enzyme was induced by fusing maltose binding protein (MBP) to Enhygromyxa salina-derived lipoxygenase, which resulted in improved reaction rate of the enzyme. That is, the result means that the dioxygenation reaction of the recombinant E. coli-based whole-cell catalyst was well performed at a rate of 665 U/g dry cells for a high-concentration of linoleic acid, and as a result, it is possible to produce 9-hydroperoxyoctadecadienoic acid or 9-hydroxyoctadecadienoic acid from linoleic acid in a high concentration.

The results of recombinant E. coli-based whole-cell catalyst biotransformation using 10 and 100 mM linoleic acids confirmed in Example were shown in Table 3. The product concentration, especially the product concentration when the substrate was 100 mM, was at least 4 times higher than that of the recombinant E. coli pET21a-EsLOX catalyst expressing EsLOX.

TABLE 3

| | Substrate | Initial | Transfor- | Product |
|---|---|---|---|---|
| | concen- | biotransfor- | mation | concen- |
| | tration | mation rate | rate | tration |
| | (mM) | (U/g dry cells)¹ | (%)² | (mM) |
| xample 3-2 | 10 | 275 | 90 | 9.8 |
| | 100 | 665 | 79 | 79 |

### Example 4. Preparation of 8S-hydroxy-(9E,11Z)-heptadecadiene(8-HHD) From Linoleic Acid Using Recombinant E. coli

4-1. Construction of Whole-Cell Catalysts Based on Recombinant Plasmid and E. coli

In the present invention, in order to prepare secondary fatty alcohol 8-HHD, which was a novel substance, from linoleic acid, recombinant E. coli was constructed by co-introducing an Enhygromyxa salina-derived lipoxygenase gene; and a Chlorella variabilis-derived decarboxylase gene catalyzing a light-dependent decarboxylation reaction.

Specifically, a vector pET28a-Cv-FAP was constructed by introducing a Cv-FAP gene (SEQ ID NO: 11) as decarboxylase catalyzing a photodecarboxylation reaction, into a pET28a vector. The vector pACYC-EsLOX constructed in Example 1-1; and a vector pET28a-Cv-FAP were transformed to E. coli BL21 (DE3). The constructed recombinant E. coli pACYC-EsLOX/pET28a-Cv-FAP was grown in the LB medium containing 50 g/mL of kanamycin and 30 μg/mL of chloramphenicol.

4-2. Preparation of 8-HHD from Linoleic Acid

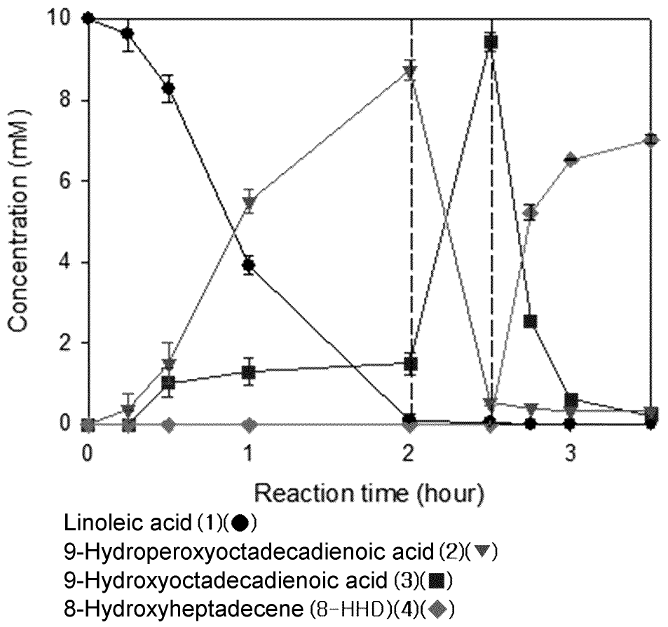

8-HHD was prepared from linoleic acid using whole-cell biocatalysts based on the recombinant E. coli pACYC-EsLOX/pET28a-CvFAP constructed in Example 4-1. Specifically, the constructed recombinant E. coli pACYC-EsLOX/pET28a-CvFAP was cultured in a terrific medium at 37° C.. After inducing gene expression with IPTG, the recombinant E. coli was cultured at 16° C. for 22 hours. Thereafter, hydroperoxy fatty acids were produced using 10 mM linoleic acid in a 50 mM EPPs buffer with 14.4 g/L of dried E. coli cells. Then, in order to reduce the hydroperoxy fatty acid, the reaction solution was added with a reducing agent (TCEP) at a concentration (20 mM) twice or more than the substrate and reacted for 20 minutes. Thereafter, the whole-cell catalytic reaction was performed under a blue LED light with a 450 nm wavelength band for light-dependent decarboxylation reaction. The biotransformation reaction was performed at 200 rpm and 30° C. in a flask in a shaking incubator, and the decarboxylation reaction was performed at 37° C. in a heating mantle. The biotransformation results were illustrated in FIG. 13.

As illustrated in FIG. 13, it was confirmed that in the recombinant E. coli pACYC-EsLOX/pET28a-CvFAP, 80% or more of the linoleic acid was transformed to 9-hydroperoxyoctadecadienoic acid (9-HpODE)(2) only in 120 minutes in the first step of 8-HHD production. Thereafter, as a result of reduction by adding a reducing agent (second step), it was confirmed that 9-hydroperoxyoctadecadienoic acid(2) was transformed to 9 mM 9-hydroxyoctadecadienoic acid(3). As a result of the light-dependent decarboxylation reaction, 7.5 mM 8-HHD(4) as secondary fatty alcohol was obtained from 10 mM linoleic acid. That is, it was confirmed that it took 3 hours and 20 minutes to produce 8-HHD from linoleic acid, and the transformation rate was 75%.

### 4-3. Purification and NMR Analysis of Reaction Product

In order to confirm the components of a sample prepared in Example 4-2, nuclear magnetic resonance (NMR) analysis was performed. The sample was purified using silica gel column chromatography for high-purity extraction of 90% or more. An NMR spectrum was analyzed using a Bruker AVIII400 instrument, and measured by dissolving TMS (trimethylsilane) in CDCl₃ and DMSO contained as an internal standard (1 H at 400 MHz, 13 C at 100 MHz).

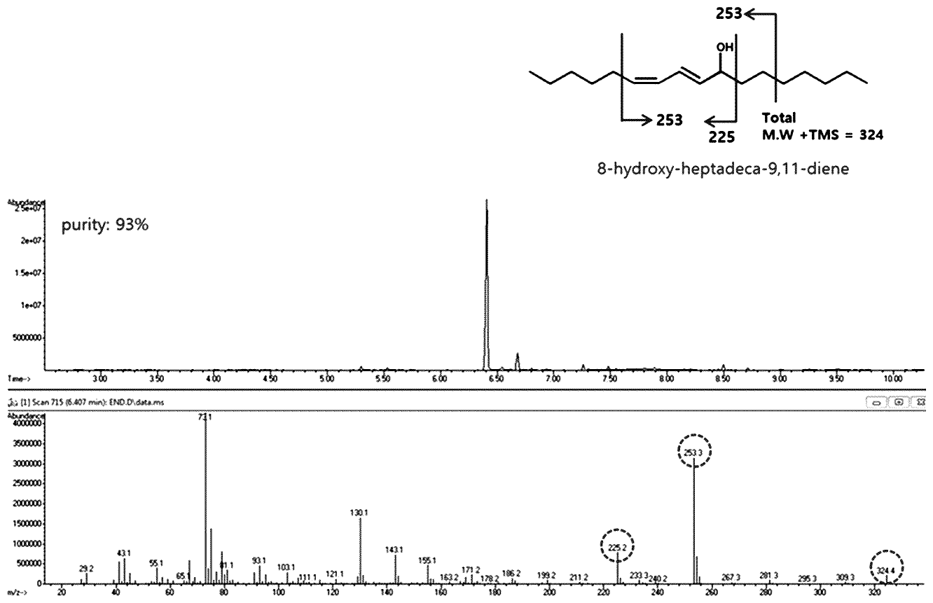

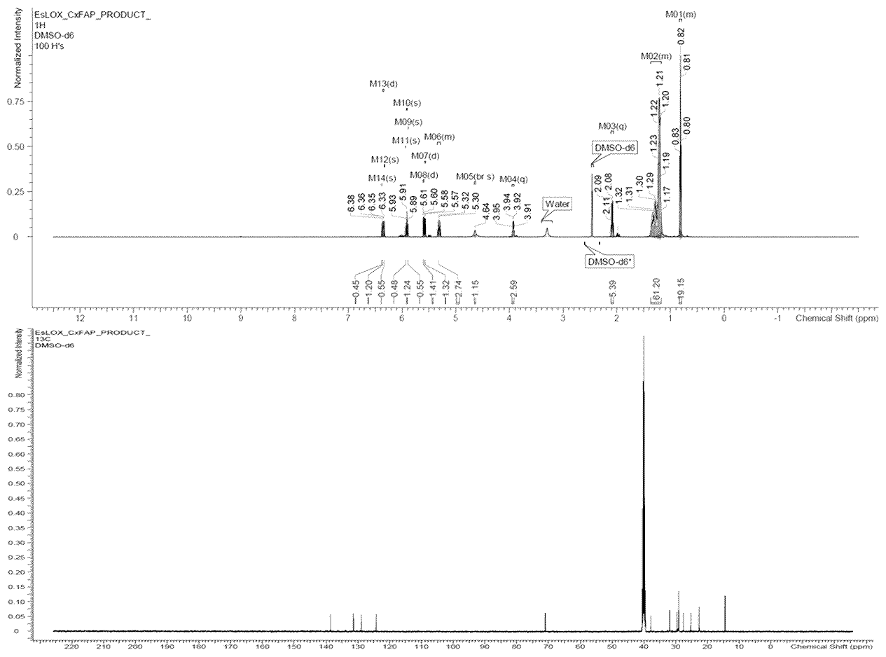

FIG. 14 is a result of analyzing the purity of the sample of Example 4 by GC/MS after purification by silica gel column chromatography, and FIG. 15 is NMR data obtained during the analysis of the sample of Example 4.

As illustrated in FIGS. 14 and 15, it can be seen that target 8-hydroxyheptadecene (8-HHD) was synthesized. As a 1H NMR result, ¹H NMR (500 MHz, DMSO-d6) δ: 6.38-6.33 (m, 1H), 5.93-5.89 (m, 1H), 5.61-5.57 (m, 1H), 5.34-5.29 (m, 1H), 4.65-4.64 (d, J=4.58 Hz, 1H), 3.95-3.91 (m, 1H), 2.11-2.06 (m, 2H), 1.35-1.17 (m, 18H), and 0.83-0.80 (m, 6H) were analyzed. In addition, as a ¹³C NMR analysis result, ¹³C NMR (125 MHz, DMSO-d6) δ: 138.61 (C7=C8-C9=C10), 131.40 (C7=C8-C9=C10), 128.92 (C7=C8-C9=C10), 124.28 (C7=C8-C9=C10), 71.03 (C—OH), 37.78, 31.87, 31.81, 29.65, 29.08, 27.59, 25.23, 22.68, 22.62, 14.48 (CH3) were confirmed to confirm the structure of the product.

Accordingly, the result means that an E. coli-based whole-cell catalyst co-expressing an Enhygromyxa salina-derived lipoxygenase catalyzing the dioxidation reaction; and a Chlorella variabilis-derived decarboxylase catalyzing light-dependent decarboxylation reaction may produce 8-hydroxyheptadecene (8-HHD) as secondary fatty alcohol from linoleic acid.

Overall, the present inventors discovered the Enhygromyxa salina-derived lipoxygenase and analyzed the characteristics thereof, and constructed recombinant E. coli containing the enzyme. The present inventors constructed the MBP-EsLOX fusion enzyme for improved water-solution expression and confirmed that when the enzyme was expressed in E. coil to be used as the whole-cell biocatalyst, high-concentration of hydroxy fatty acids may be produced from unsaturated fatty acids such as linoleic acid at a fast rate. In addition, the present inventors constructed a recombinant strain co-expressing the Enhygromyxa salina-derived lipoxygenase and the Chlorella variabilis-derived decarboxylase, and confirmed that when the recombinant strain was used as a whole-cell biocatalyst, secondary fatty alcohols may be produced from unsaturated fatty acids such as linoleic acid. The recombinant E. coli constructed in the present invention can be used in various ways in the production field of hydroxy fatty acids and secondary fatty alcohols.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1.-37. (canceled)

38. A method for producing hydroxy fatty acids, comprising preparing hydroperoxy fatty acids by reacting recombinant microorganisms with unsaturated fatty acids,
wherein the recombinant microorganisms comprise a lipoxygenase gene represented by at least one nucleotide sequence selected from the group consisting of SEQ ID NOs: 1, 2, 8, 9 and 10.

39. The method for producing the hydroxy fatty acids of claim 38, wherein the lipoxygenase gene is derived from Pseudomonas aeruginosa.

40. The method for producing the hydroxy fatty acids of claim 38, wherein the lipoxygenase is secreted into periplasm or cytoplasm.

41. The method for producing the hydroxy fatty acids of claim 38, wherein the recombinant microorganisms are for producing hydroxy fatty acids or hydroperoxy fatty acids.

42. The method for producing the hydroxy fatty acids of claim 38, wherein the recombinant microorganisms are for whole-cell biotransformation.

43. The method for producing the hydroxy fatty acids of claim 38, further comprising:
reducing the hydroperoxy fatty acids.

44. The method for producing the hydroxy fatty acids of claim 38, wherein the unsaturated fatty acids are at least one selected from the group consisting of linoleic acid, α-linolenic acid, γ-linolenic acid, arachidonic acid, oleic acid and palmitoleic acid.

45. The method for producing the hydroxy fatty acids of claim 38, wherein the hydroxy fatty acids are at least one selected from the group consisting of 13-hydroxyoctadecadienoic acid, 13-hydroxyoctadecatrienoic acid, and 15-hydroxyeicosatetraenoic acid.

46. The method for producing the hydroxy fatty acids of claim 38, wherein the recombinant microorganisms further comprise a decarboxylase gene represented by a gene set forth in SEQ ID NO: 3 or 11.

47. The method for producing the hydroxy fatty acids of claim 46, wherein the decarboxylase is derived from Chlorella variabilis.

48. The method for producing the hydroxy fatty acids of claim 46, wherein the recombinant microorganisms are for producing hydroxy fatty acids, hydroperoxy fatty acids, or secondary fatty alcohols.

49. The method for producing the hydroxy fatty acids of claim 48, wherein the secondary fatty alcohols are at least one selected from the group consisting of linoleic acid-derived fatty alcohol 6-hydroxy-7,9-heptadecene, γ-linolenic acid-derived fatty alcohol 6-hydroxy-7,9,12-heptadecatriene, α-linolenic acid-derived fatty alcohol 6-hydroxy-3,7,9-heptadecatriene, and oleic acid-derived fatty alcohol 9-hydroxy-8-heptadecene.

50. The method for producing the hydroxy fatty acids of claim 38, wherein the lipoxygenase gene is derived from Enhygromyxa salina.

51. The method for producing the hydroxy fatty acids of claim 38, wherein the recombinant microorganisms further include a maltose binding protein (MBP)-coding sequence represented by a nucleotide sequence set forth in SEQ ID NO: 18.

52. The method for producing the hydroxy fatty acids of claim 38, wherein the hydroxy fatty acids are at least one selected from the group consisting of 9-hydroxyoctadecadienoic acid and 9-hydroxyoctadecatrienoic acid.

53. Recombinant microorganisms comprising a lipoxygenase gene represented by at least one nucleotide sequence selected from the group consisting of SEQ ID NOs: 1, 2, 8, 9 and 10.

54. Recombinant microorganisms comprising a lipoxygenase gene represented by a nucleotide sequence set forth in SEQ ID NO: 1 or 2.

55. A method for preparing recombinant microorganisms for producing hydroxy fatty acids, comprising introducing a lipoxygenase gene into microorganisms,
wherein the lipoxygenase gene is at least one nucleotide sequence selected from the group consisting of SEQ ID NOs: 1, 2, 8, 9 and 10.


SEQUENCE LISTING (18 sequences)

SEQ ID NO: 1 - DNA, 2040 bp, Pseudomonas aeruginosa
atgcatcaccatcatcaccacagccaggatccgaattcgaatgactcgatattcttttcacccttgaaatacctcggggcggaacaacagcgatctatcgatgcctcgcgaagtttgctggacaacctgattccgccgagtttgccgcaatatgacaacctggccggaaagttggcgcggcgtgcggtattgaccagcaagaaactggtatatgtctggaccgagaacttcggcaatgtcaaaggcgtgccgatggcccgctcggtgccgctgggcgagttgccgaatgtcgactggctgttgaagaccgccggggtgatcgtcgagctgatcgtcaatttcgtcgcctcgttgccggcgtcggccgcagcgcagttcgagcggatcgccaccgggctcagcggtgacctggaggcggcgcgccaggtccacgaggcgttgctggaggaggcgaagaacgatccggccgcggccggcagcctgctgctgcgcttcacggagttgcagacgcgggtgatcgccatactgacgcgggtcggcctgctggtcgacgatattctcaagagcgccagcaacctggttacccagagggggcagggcgacggcctgaatcgcttccgcgcggtgttcggcaccctgcgcctgccggaggtcgccgacagcttccgtgacgacgaagcgttcgcctactggcgggtggccgggcctaatccgctgctgatccgtcgcgtcgatgcgctgccggccaactttccgctgggcgaggagcagttccgccgggtgatgggcgccgacgacagcttgctggaggccgccgccagccgccgcctgtatctgctggactacgccgagctgggcaagctggcgccgtcgggagcggtggacaaactgctcaccgggacgggcttcgcctatgcgccgatcgccctgttcgcccttggcaaggatcgggcgaggctattgccggtggccatccagtgcggtcaggaccccgctacccatccgatgttcgtgcgtcccgccgagtcggagagtgatctgtactggggttggcagatggccaagacggtggtccaggtcgccgaggagaactaccacgagatgttcgtccacctggcccagacccacctggtgagcgaggcgttctgcctggccacccagcgcaccctggcgcccagccatccgctgcacgtcctgctcgccccgcacttcgagggcaccctgttcatcaacgagggggcggcgcggatcctgttgcccagcgcgggcttcatcgacgtgatgttcgccgcgccgatccaggacacccaggccaccgccggcggcaatcggctgggtttcgacttctaccgcggcatgttgccggagagcctgaaggcgcggaacgtcgacgacccgctggcgctaccggactacccctatcgcgacgacggcctgctggtatggaatgccatccgccagtgggcggccgactacgtggcggtgtactacgccagcgacggcgacgtcaccgctgacgtcgaactggcggcctgggtcggcgaagtgatcggcagcggcaaggtggccgggttccgcccgatcaccggtcgcagccaactggtcgaggtgctgaccatggtgatcttcactgcctccgcgcagcatgcggcggtcaattttccccagccttcgatgatgacctacgcaccagccatctgtgccatgagcgcggcgccggcaccggactcgccaagcggcaagagcgaggccgattggttgaagatgatgccgccgaccctggtggcgctggagaaggtgaacatctatcacctgctcggttcggtctaccacggtcgcctgggcgactaccggcagaccgggttcccctacgcgccggtgttttcggacaggcgggtgaccgccagcggcggcccgctggagcgcttccaggcacgcctgaaggaggtcgaggcgaccattcgcacgcgcaaccaggcgcgccgcaggccctacgagtacctgttgccgagccggatcccggcgagcaccaatatctga

SEQ ID NO: 2 - DNA, 2049 bp, Pseudomonas aeruginosa
atgggcagcagccatcaccatcatcaccacagccaggatccgaattcgaatgactcgatattcttttcacccttgaaatacctcggggcggaacaacagcgatctatcgatgcctcgcgaagtttgctggacaacctgattccgccgagtttgccgcaatatgacaacctggccggaaagttggcgcggcgtgcggtattgaccagcaagaaactggtatatgtctggaccgagaacttcggcaatgtcaaaggcgtgccgatggcccgctcggtgccgctgggcgagttgccgaatgtcgactggctgttgaagaccgccggggtgatcgtcgagctgatcgtcaatttcgtcgcctcgttgccggcgtcggccgcagcgcagttcgagcggatcgccaccgggctcagcggtgacctggaggcggcgcgccaggtccacgaggcgttgctggaggaggcgaagaacgatccggccgcggccggcagcctgctgctgcgcttcacggagttgcagacgcgggtgatcgccatactgacgcgggtcggcctgctggtcgacgatattctcaagagcgccagcaacctggttacccagagggggcagggcgacggcctgaatcgcttccgcgcggtgttcggcaccctgcgcctgccggaggtcgccgacagcttccgtgacgacgaagcgttcgcctactggcgggtggccgggcctaatccgctgctgatccgtcgcgtcgatgcgctgccggccaactttccgctgggcgaggagcagttccgccgggtgatgggcgccgacgacagcttgctggaggccgccgccagccgccgcctgtatctgctggactacgccgagctgggcaagctggcgccgtcgggagcggtggacaaactgctcaccgggacgggcttcgcctatgcgccgatcgccctgttcgcccttggcaaggatcgggcgaggctattgccggtggccatccagtgcggtcaggaccccgctacccatccgatgttcgtgcgtcccgccgagtcggagagtgatctgtactggggttggcagatggccaagacggtggtccaggtcgccgaggagaactaccacgagatgttcgtccacctggcccagacccacctggtgagcgaggcgttctgcctggccacccagcgcaccctggcgcccagccatccgctgcacgtcctgctcgccccgcacttcgagggcaccctgttcatcaacgagggggcggcgcggatcctgttgcccagcgcgggcttcatcgacgtgatgttcgccgcgccgatccaggacacccaggccaccgccggcggcaatcggctgggtttcgacttctaccgcggcatgttgccggagagcctgaaggcgcggaacgtcgacgacccgctggcgctaccggactacccctatcgcgacgacggcctgctggtatggaatgccatccgccagtgggcggccgactacgtggcggtgtactacgccagcgacggcgacgtcaccgctgacgtcgaactggcggcctgggtcggcgaagtgatcggcagcggcaaggtggccgggttccgcccgatcaccggtcgcagccaactggtcgaggtgctgaccatggtgatcttcactgcctccgcgcagcatgcggcggtcaattttccccagccttcgatgatgacctacgcaccagccatctgtgccatgagcgcggcgccggcaccggactcgccaagcggcaagagcgaggccgattggttgaagatgatgccgccgaccctggtggcgctggagaaggtgaacatctatcacctgctcggttcggtctaccacggtcgcctgggcgactaccggcagaccgggttcccctacgcgccggtgttttcggacaggcgggtgaccgccagcggcggcccgctggagcgcttccaggcacgcctgaaggaggtcgaggcgaccattcgcacgcgcaaccaggcgcgccgcaggccctacgagtacctgttgccgagccggatcccggcgagcaccaatatctga

SEQ ID NO: 3 - DNA, 2178 bp, Unknown
  Chlorella variabilis
atgaaatcttctcaccatcaccatcaccatggttcttctatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccgggatcgaggaaaacctgtacttccaatccgcgtctgccgttgaagacatccgtaaagtcctgtccgattcttcgtctccggtggcgggtcagaaatatgactacatcctggttggcggtggcaccgcggcgtgcgtgctggcaaaccgtctgagcgctgacggttccaaacgtgtactggttctggaagcaggcccggataacacctcccgcgacgttaagattccggcggcgatcacccgcctgttccgctccccgctggactggaacctgttctctgaactgcaggaacagcttgcggaacgtcagatctacatggcgcgtggccgtctgctgggcggttccagcgcgactaacgccactctgtaccaccgtggtgcggcgggtgattacgacgcatggggtgttgaaggctggtccagcgaagacgttctgtcttggttcgtccaggcggaaaccaacgcggacttcggtccgggcgcttatcatggcagcggcggcccgatgcgtgtggaaaacccgcgttacaccaacaaacagctgcacactgctttcttcaaggctgctgaagaagttggtcttaccccgaactccgatttcaacgattggagccatgaccacgccggttacggcacctttcaggtgatgcaggataaaggcacccgcgcggatatgtaccgtcagtatctgaaacctgtgctgggtcgtcgcaacctgcaggtactgaccggcgctgcagtgaccaaagtcaacatcgaccaggctgcgggcaaagcgcaggctctgggtgttgaattctccaccgacggcccaaccggcgaacgcctgtctgcggaactggctccgggtggtgaggtcatcatgtgcgcaggtgctgttcacaccccgttcctgctgaaacattccggcgttggcccgtctgctgagctgaaagaattcggcatcccggttgttagcaacctggctggtgttggccagaacctgcaggatcagccggcgtgcctgaccgcggctccggttaaagaaaaatacgacggtattgccatttctgatcacatctacaacgaaaaaggccagatccgtaaacgtgcaatcgcatcctacctgctgggtggtcgtggcggtctgacttccaccggttgcgatcgcggtgccttcgttcgtaccgcgggtcaggcgctgccggacctgcaggttcgcttcgttccaggtatggcgctggacccggacggtgttagcacctacgttcgttttgctaaattccagagccagggtctgaaatggccgagcggcatcaccatgcagctgatcgcttgccgtccgcagtctaccggctccgtcggtcttaaatccgctgacccgtttgcgccgccgaaactgtcaccaggttacctgaccgacaaagacggtgctgatctggctaccctgcgtaaaggcatccattgggcacgtgatgttgcgcgtagctctgctctgtccgaatacctggatggtgagctgttcccaggtagcggcgttgtttctgatgatcagatcgatgaatatatccgtcgttctatccactcgtccaacgctatcactggcacctgtaaaatgggtaacgcaggtgacagcagctctgtggtagacaaccagctgcgtgttcacggtgttgaaggcctgcgcgttgttgacgctagcgttgttccgaaaattccgggtggtcagaccggtgcgccggtagttatgatcgctgaacgcgcagcagctctgctgacggggaaagcaaccattggtgcatctgctgctgcaccggcgaccgtagctgcataa

SEQ ID NO: 4 - DNA, 32 bp, Artificial
  pET22b-Pa-LOX forward primer
cggcgatggccatgcatcaccatcatcaccac

SEQ ID NO: 5 - DNA, 31 bp, Artificial
  pET22b-Pa-LOX reverse primer
gcggccgcaagctttcagatattggtgctcg

SEQ ID NO: 6 - DNA, 39 bp, Artificial
  pACYC-Pa-LOX forward primer
gccaggatccgaattcgaatgactcgatattcttttcac

SEQ ID NO: 7 - DNA, 32 bp, Artificial
  pACYC-Pa-LOX reverse primer
atgcggccgcaagctttcagatattggtgctc

SEQ ID NO: 8 - DNA, 1953 bp, Artificial
  Enhygromyxa salina-derived lipoxygenase_pET21a
atgtccaacatcccaacgctccctcaaaatgatccccgccccgagcaacgagcccagcagctcgacaaggcccgggagacctacaaatacgcggatctgctgcccccgctggccttctgtgagggggtccccaagcccgacacgccaagcgcggcgtggctggtcaccgttggcaaggtcgcggccgcggtcgcattgaatgcagtcgccaaccgccgggcctggaagcgcggcgatcacgacgaggaggacgcatcgttcgatgtgggcggcaccagcgaagaagaccagaacgaggccggtcacaacagcttcttggcgcggctcgagaacttcgatgaagaggcggtctcgttcgccccgggcgacgagcccagcgtgctcgacgaggtcaacgagcgcgtcgccgcgatcttgggggccaagcccaaccgtcacgtgccgccggcccaagccgacgacgatgacaacggcgacgacggcgacggcgacgagagcttcggcgtgctcgacggcaacgtgtccaaggacggccccaacggccgcccgcagagcatggacgactacgccaacttgtttcggcgcatcacgctgccgccgatcgccagcacctggaagaacgactcggcgttcgcggcctaccgcgtggccgggcccaacgccagcatgatccagcggatcaccgagctgcccgacaacttcgcggtcaccgacgcccactacaaacaagcaatgggcgagggtgactcgctcgacgcggccaaggccgaaggtcggttgttcctggccgactggaagctgatcggtgagaccctggtcaacaacacctacaagggcgcccagaagaccgtgtacgcgccgctggccctgttcgcggtcccgccgggcggtggctcgctggcgcccgtggcgatccaaccaggccagaccccaagcccaacgaacaagatctacgcgacccaggatggcaacgactggctcgcggccaagagcgccgtgcaggtcgcggagggcaactatcacgagctcgtcagccacctcggcctgacccatctgctgctcgagccgatcgtgatggcgacctatcgccagctcgcgcaacaccaccccatctacatgctgctgatcccccacttcgagggcacgctgtcgatcaacaacagcgcggcgaccaacctgatcgccccgggtggggccgttgacctgatcttcgctggcacgatcgagtccgagcaccagctggccctggccgccctgaagcgccacgacttcatgcgctcgggcctgcccgacacgatcgagcagcgcggcgtcggcgataccagcgtgctgaccgactacccctatcgcgacgacggcctgaagatctgggccaacatcgagcgctgggtcacggcctacgtcaacaactactacatcagcgaggccaacgtcacccaagacacagagctgcaggcgtgggcggccgtgctcagcaagcccttcgccgagggcggcgtgtctgggtttggcccgatcgacacccgggccgcgctgatcttcgcgtgcacgaaggtcatcttcacggccagcgccgagcactcggccgtcaacttcccgcagaaggatctgatgtcctacgcgcccgcgatcaccggcgcgggctggaccgctgcgccgccctcacaaggcccgctgaaggacttccagccgccgctcgagctcgccgagctgcaggccgagttcctgtatttgctgggcggcgtccaccacaccaagctgggcttctacaactccaacagcttcccgtaccgggcctggttcaaggatcccaagatcaccgccgagctgctgcccgcgttccagcgggacctcgcggccagcgaagagctgatcgtcgcggccaacgccacccgcacgttcaagtacacgtacatgatcccgtccaccattccgatgagcatcaacatctga

SEQ ID NO: 9 - DNA, 1953 bp, Artificial; identical to SEQ ID NO: 8
  Enhygromyxa salina-derived lipoxygenase_pACYC

SEQ ID NO: 10 - DNA, 3150 bp, Artificial
  Enhygromyxa salina-derived lipoxygenase_pB4
atgggcagcagcaatcatcatcatcatcacggtaccaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggtaccgattacgatatcccagcgaccgaaaacctttacttccagggcggcggtggtggcggcatgtccaacatcccaacgctccctcaaaatgatccccgccccgagcaacgagcccagcagctcgacaaggcccgggagacctacaaatacgcggatctgctgcccccgctggccttctgtgagggggtccccaagcccgacacgccaagcgcggcgtggctggtcaccgttggcaaggtcgcggccgcggtcgcattgaatgcagtcgccaaccgccgggcctggaagcgcggcgatcacgacgaggaggacgcatcgttcgatgtgggcggcaccagcgaagaagaccagaacgaggccggtcacaacagcttcttggcgcggctcgagaacttcgatgaagaggcggtctcgttcgccccgggcgacgagcccagcgtgctcgacgaggtcaacgagcgcgtcgccgcgatcttgggggccaagcccaaccgtcacgtgccgccggcccaagccgacgacgatgacaacggcgacgacggcgacggcgacgagagcttcggcgtgctcgacggcaacgtgtccaaggacggccccaacggccgcccgcagagcatggacgactacgccaacttgtttcggcgcatcacgctgccgccgatcgccagcacctggaagaacgactcggcgttcgcggcctaccgcgtggccgggcccaacgccagcatgatccagcggatcaccgagctgcccgacaacttcgcggtcaccgacgcccactacaaacaagcaatgggcgagggtgactcgctcgacgcggccaaggccgaaggtcggttgttcctggccgactggaagctgatcggtgagaccctggtcaacaacacctacaagggcgcccagaagaccgtgtacgcgccgctggccctgttcgcggtcccgccgggcggtggctcgctggcgcccgtggcgatccaaccaggccagaccccaagcccaacgaacaagatctacgcgacccaggatggcaacgactggctcgcggccaagagcgccgtgcaggtcgcggagggcaactatcacgagctcgtcagccacctcggcctgacccatctgctgctcgagccgatcgtgatggcgacctatcgccagctcgcgcaacaccaccccatctacatgctgctgatcccccacttcgagggcacgctgtcgatcaacaacagcgcggcgaccaacctgatcgccccgggtggggccgttgacctgatcttcgctggcacgatcgagtccgagcaccagctggccctggccgccctgaagcgccacgacttcatgcgctcgggcctgcccgacacgatcgagcagcgcggcgtcggcgataccagcgtgctgaccgactacccctatcgcgacgacggcctgaagatctgggccaacatcgagcgctgggtcacggcctacgtcaacaactactacatcagcgaggccaacgtcacccaagacacagagctgcaggcgtgggcggccgtgctcagcaagcccttcgccgagggcggcgtgtctgggtttggcccgatcgacacccgggccgcgctgatcttcgcgtgcacgaaggtcatcttcacggccagcgccgagcactcggccgtcaacttcccgcagaaggatctgatgtcctacgcgcccgcgatcaccggcgcgggctggaccgctgcgccgccctcacaaggcccgctgaaggacttccagccgccgctcgagctcgccgagctgcaggccgagttcctgtatttgctgggcggcgtccaccacaccaagctgggcttctacaactccaacagcttcccgtaccgggcctggttcaaggatcccaagatcaccgccgagctgctgcccgcgttccagcgggacctcgcggccagcgaagagctgatcgtcgcggccaacgccacccgcacgttcaagtacacgtacatgatcccgtccaccattccgatgagcatcaacatctga

SEQ ID NO: 11 - DNA, 2178 bp, Artificial; identical to SEQ ID NO: 3
  Chlorella variabilis-derived decarboylase

SEQ ID NO: 12 - DNA, 32 bp, Artificial; identical to SEQ ID NO: 4
  pET21a-EsLOX forward primer

SEQ ID NO: 13 - DNA, 31 bp, Artificial
  pET21a-EsLOX reverse primer
gctcgtggttatagactttcgaacgccggcg

SEQ ID NO: 14 - DNA, 32 bp, Artificial
  pACYC-EsLOX forward primer
ggagatataccatggatgaaatacctgctgcc

SEQ ID NO: 15 - DNA, 26 bp, Artificial
  pACYC-EsLOX reverse primer
cggccgcaagctttcagatgttgatg

SEQ ID NO: 16 - DNA, 30 bp, Artificial
  pB4-EsLOX forward primer
gcggtggtggcggcatgtccaacatcccaa

SEQ ID NO: 17 - DNA, 30 bp, Artificial
  pB4-EsLOX reverse primer
gccctcagatgttgatgctcagatgttgat

SEQ ID NO: 18 - DNA, 1098 bp, Artificial
  MBP(maltose binding protein)
aaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagact